(19) United States
(12) Patent Application Publication
(10) Pub. No.: US 2020/0390838 A1
(43) Pub. Date: Dec. 17, 2020

(54) APPARATUS AND METHOD FOR EXTRACTION AND DECARBOXYLATION OF PHYTOCANNABINOIDS

(71) Applicant: CANNSCIENCE INNOVATIONS INC., Toronto (CA)

(72) Inventors: Lakshmi Premakanth KOTRA, Toronto (CA); Nicholas HURLEY, Ancaster (CA); Peter B SAMPSON, Oakville (CA); Giridhar CHEEKATI, Mississauga (CA)

(21) Appl. No.: 16/956,912

(22) PCT Filed: Dec. 21, 2018
(86) PCT No.: PCT/CA2018/051653

§ 371 Date: Jun. 22, 2020

Related U.S. Application Data
(60) Provisional application No. 62/609,708, filed on Dec. 22, 2017.

(51) Int. Cl. A61K 36/185; B01D 11/02

(52) U.S. Cl. CPC A61K 36/185; B01D 11/0211; B01D 11/0288; A61K 2236/39; B01D 11/0257; A61K 2236/15; B01D 11/0207

(57) ABSTRACT

The present disclosure relates to an apparatus for the extraction and decarboxylation of phytocannabinoids. The apparatus is a high-efficiency and high volume extractor and activator for cannabis. The apparatus is engineered to accommodate batch or continuous flow mechanisms, allowing for flexibility and highly efficient extraction and decarboxylation, preferably in a single-pass. The present disclosure also relates to a continuous flow method for the extraction and decarboxylation of phytocannabinoids, preferably in a single-pass, and the products produced by the method and apparatus.

### CROSS REFERENCE TO PRIOR APPLICATIONS

This application claims priority under the Paris Convention to U.S. Provisional Patent Application Ser. No. 62/609,708 filed Dec. 22, 2017, which is incorporated herein by reference as if set forth herein in its entirety.

### FIELD OF THE DISCLOSURE

The present disclosure relates to an apparatus for the extraction and decarboxylation of phytocannabinoids. The apparatus is a high-efficiency and high volume extractor and activator for cannabis. The apparatus is engineered to accommodate batch or continuous flow mechanisms, allowing for flexibility and highly efficient extraction and decarboxylation, preferably in a single-pass. The present disclosure also relates to a continuous flow method for the extraction and decarboxylation of phytocannabinoids, preferably in a single-pass, and the products produced by the method and apparatus.

### BACKGROUND OF THE DISCLOSURE

Cannabis is a genus of flowering plants in the family Cannabaceae, and includes hemp. Three species may be recognized (Cannabis sativa, Cannabis indica and Cannabis ruderalis). Cannabis spp. contain a highly complex mixture of compounds, including phytocannabinoids.

Cannabis spp. plant material including the flower, stalk, and other plant parts that are above the surface (either soil, or water if grown using hydroponics) carry moderate to significant amounts of phytocannabinoids. Most cannabinoids are concentrated in a viscous resin produced in structures of the cannabis plant known as glandular trichomes. At least 113 different cannabinoids have been isolated from the cannabis plant. Several of these phytocannabinoids carry a carboxyl moiety, so called “acid forms” of phytocannabinoids. Δ⁹-Tetrahydrocannabinol (Δ⁹-THC), cannabidiol (CBD), and cannabigerol (CBG) are among the most common phytocannabinoids that also exist in the corresponding “acid forms”, viz. Δ⁹-tetrahydrocannabinol-2-carboxylic acid (Δ⁹-THCA), cannabidiol carboxylic acid (CBDA) and cannabigerolic acid (CBGA), respectively. These compounds along with other phytocannabinoids, terpenes, flavonoids, etc. are present in the cannabis plant. Individual phytocannabinoid concentrations may be different in different species or varieties of Cannabis.

In most cases, it is the decarboxylated form of the phytocannabinoid that is active and potent in mammals. For example, Δ⁹-THCA is not psychoactive in a human, whereas Δ⁹-THC is psychoactive and potent in a human.

Spontaneous decarboxylation of phytocannabinoids occurs when exposed to heat. The decarboxylated phytocannabinoids are desired compounds. Plant material consumed after the phytocannabinoids are decarboxylated exhibits higher potency. It is also desirable to promote decarboxylation of phytocannabinoids in the extracts of cannabis, such that the products prepared from such cannabis extracts will exhibit high potency.

Completion of the decarboxylation of phytocannabinoids in the plant material and/or cannabis extract without leaving any natural acid forms in the fully-decarboxylated cannabis extract is of high value. Such a material, when one can prepare repeatedly with a high level of consistency, reproducibility and scalability, provides industry the ability to employ in pharmaceutical, medical and recreationally-oriented products. Standardized methods taking advantage of devices/equipment designed to manufacture or produce such materials also pave way for the implementation for industry-standard manufacturing to produce cannabis resin (cannabis extract) with fully-decarboxylated phytocannabinoids.

### SUMMARY OF THE DISCLOSURE

This invention relates to:

<1> An apparatus for extracting and decarboxylating cannabinoids to produce a decarboxylated cannabis product, the apparatus comprising:

one or more inputs;
one or more heating tubes;
one or more outputs; and
one or more heating mechanisms;
the one or more inputs for accepting a cannabis input under pressure, the cannabis input containing the cannabinoids;
wherein, in use, one(s) of said one or more heating mechanisms raises the temperature of the cannabis input to a desired temperature, and other(s) of said one or more heating mechanisms maintains the cannabis input at the desired temperature for a desired time to effect an amount of decarboxylation, and
said cannabis input continuously flows from the one or more inputs to the one or more outputs while being subjected to heat by said one or more heating mechanisms at a flow rate sufficient to effect the amount of decarboxylation in a single pass through the apparatus, to produce at said one or more outputs said decarboxylated cannabis product.

<2> The apparatus of <1> wherein the amount of decarboxylation is 92% to 100%.

<3> The apparatus of <1> wherein the amount of decarboxylation is 94% to 100%.

<4> The apparatus of <1> wherein the amount of decarboxylation is 96% to 100%.

<5> The apparatus of <1> wherein the amount of decarboxylation is 98% to 100%.

<6> The apparatus of <1> wherein the amount of decarboxylation is 99% to 100%.

<7> The apparatus of <1> wherein the amount of decarboxylation is 100%.

<8> The apparatus of <1> wherein said one(s) of said one or more heating mechanisms comprises a microwave generator.

<9> The apparatus of <1> wherein said one(s) of said one or more heating mechanisms comprises a flame.

<10> The apparatus of <1> wherein the cannabis input comprises an organic solvent.

<11> The apparatus of <10> wherein the organic solvent comprises a pharmaceutically acceptable, reagent-grade, food-grade, or pharmaceutical grade solvent.

<12> The apparatus of <10> wherein the organic solvent comprises ethanol or isopropanol.

<13> The apparatus of <10> wherein the organic solvent has a boiling point less than 100° C.

<14> The apparatus of <1> wherein the flow rate is about 5 mL/minute to about 15 L/minute.

<15> The apparatus of <8> wherein one of said heating tubes comprises a reactor tube, wherein said cannabis input is irradiated by microwave radiation to a temperature of about 135° C. to about 200° C. by said microwave generator while traversing said reactor tube.

<16> The apparatus of <15> wherein said reactor tube comprises quartz.

<17> The apparatus of <16> wherein the quartz reactor tube has an interior diameter of about 4 mm to about 120 mm and a length of about 30 cm to about 200 cm.

<18> The apparatus of <15> further comprising a holding pipe adjacent and contiguous to the reactor tube, said holding pipe for maintaining said cannabis input exiting the reactor tube at said desired temperature, said other(s) of said one or more heating mechanisms for heating the holding pipe.

<19> The apparatus of <18> wherein said other(s) of said one or more heating mechanisms comprise thermal or infrared radiation.

<20> The apparatus of <19> wherein said holding pipe is thermally insulated.

<21> The apparatus of <18> further comprising two or more receiver vessels for collecting said decarboxylated cannabis product from said holding pipe.

<22> The apparatus of <21> further comprising a cooling mechanism for receiving the cannabis input from the holding pipe to cool the cannabis input to less than 60° C., less than 50° C., or less than 45° C. prior to the cannabis input collection in the two or more receiver vessels.

<23> The apparatus of <9> wherein one of said heating tubes comprises a reactor tube, wherein said cannabis input is heated by said flame to a temperature of about 135° C. to about 200° C. while traversing said reactor tube.

<24> The apparatus of <22> wherein the reactor tube comprises steel, brass or other non-reactive metal conductor.

<25> A method for decarboxylating cannabinoids, comprising:

passing (a) a suspension of plant material comprising cannabinoids in a solvent or (b) a solution of cannabinoids through a continuous flow microwave apparatus, wherein the apparatus heats the suspension or solution to 135° C.-200° C. under pressure of 10-25 Bar for sufficient time to decarboxylate the cannabinoids and produce decarboxylated cannabinoids.

<26> A method for decarboxylating cannabinoids, comprising:

passing (a) a suspension of plant material comprising cannabinoids in a solvent or (b) a solution of cannabinoids through the apparatus of any one of <1>-<24>, wherein the apparatus heats the suspension or solution under pressure and decarboxylates the cannabinoids to produce decarboxylated cannabinoids.

<27> The method of any one of <25>-<26>, wherein the plant material is cannabis plant material.

<28> The method of any one of <25>-<27>, wherein the method further comprises (i) breaking down the plant material before placing the plant material in the solvent to produce the suspension; and (ii) grinding the plant material in the solvent of the suspension,

wherein the cannabinoids are extracted from the plant material into the solvent of the suspension during passage through the apparatus.

<29> The method of any one of <25>-<28>, wherein the method further comprises a step of processing the decarboxylated cannabinoids into a decarboxylated resin after the step of passing the suspension or solution through the apparatus.

<30> The method of any one of <25>-<29>, wherein the resident time of the cannabis suspension or cannabis solution is between 45-75 minutes.

<31> The method of any one of <25>-<30>, wherein the plant material is a cannabis trichome, cannabis female inflorescence, a cannabis flower bract, a cannabis stalk, a cannabis leaf or combinations thereof.

<32> The method of any one of <25>-<28> wherein the decarboxylated cannabinoids are recovered in the form of isolated compounds.

<33> The method of any one of <25>-<32> wherein the plant material is dried cannabis.

<34> The method of any one of <25>-<33>, wherein the cannabinoids are between 92-100% decarboxylated.

<35> A product comprising decarboxylated cannabinoids produced by the method of any one of <25>-<35>, wherein the cannabinoids are between 92-100% decarboxylated.

<36> Use of the apparatus of any one of <1>-<24> for effecting any chemical reaction requiring high heat and pressure.

### BRIEF DESCRIPTION OF THE DRAWINGS

In order that the subject matter may be readily understood, embodiments are illustrated by way of examples in the accompanying drawings, in which:

FIG. 1 is a schematic diagram of an example embodiment of an apparatus described herein;

FIG. 2 is a side view of a portion of an example embodiment of an apparatus described herein, showing an example configuration of connectors/ports;

FIG. 3 is a diagram showing an example embodiment of various valves connected to receiver vessels of an example apparatus described;

FIG. 4 is a partial front view of an example embodiment of an apparatus described herein, showing receiver vessels;

FIG. 5 depicts an example Human Machine Interface (HMI) Logon screen of an example embodiment of an apparatus described herein;

FIG. 6 depicts an example Human Machine Interface (HMI) of an example embodiment of an apparatus described herein;

FIG. 7 is a conceptual diagram showing multiple reactor tubes of an example embodiment of an apparatus described herein;

FIG. 8 is a perspective view of an example embodiment of an apparatus described herein;

FIG. 9 is a rear view of an example embodiment of an apparatus described herein;

FIG. 10 is a left-side view of an example embodiment of an apparatus described herein;

FIG. 11 is a perspective view of a frame assembly of an example embodiment of an apparatus described herein;

FIG. 12 is a perspective view of an enclosure assembly of an example embodiment of an apparatus described herein;

FIG. 13 is a perspective view of a holding pipe of an example embodiment of an apparatus described herein;

FIG. 14A is a perspective view of a nitrogen feed assembly of an example embodiment of an apparatus described herein;

FIG. 14B is a perspective view of a nitrogen exhaust assembly of an example embodiment of an apparatus described herein;

FIG. 15 is a perspective view of a microwave assembly of an example embodiment of an apparatus described herein;

FIG. 16 is a perspective view of a feed assembly of an example embodiment of an apparatus described herein;

FIG. 17 is a perspective view of an upper manifold of an example embodiment of an apparatus described herein;

FIG. 18A is a perspective view of a lower bulkhead of an example embodiment of an apparatus described herein;

FIG. 18B is a front view of the lower bulkhead of FIG. 18A;

FIG. 18C is a front view of another example lower bulkhead of an example embodiment of an apparatus described herein;

FIG. 19 is a perspective view of receiver vessels of an example embodiment of an apparatus described herein;

FIG. 20A is a perspective view of a cooling mechanism of an example embodiment of an apparatus described herein;

FIG. 20B is a perspective view of a cooling mechanism outlet of an example embodiment of an apparatus described herein;

FIG. 20C is a perspective view of a cooling mechanism inlet of an example embodiment of an apparatus described herein;

FIG. 21 is a flow diagram of an example method described herein;

FIG. 22 is a flow diagram of a further example method described herein;

FIG. 23 is a flow diagram of a further example method described herein;

FIG. 24 is a flow diagram of a further example method described herein;

FIG. 25 is a flow diagram of a further example method described herein;

FIG. 26 is a flow diagram of a further example method described herein;

FIG. 27 is a flow diagram of a further example method described herein;

FIG. 28 is a flow diagram of a further example method described herein;

FIG. 29 is a flow diagram of a further example method described herein;

FIG. 30 is a flow diagram of a further example method described herein;

FIG. 31 is a flow diagram of a further example method described herein;

FIG. 32 is a flow diagram of yet a further example method described herein; and

FIG. 33 depicts a schematic diagram of an example controller described herein.

### DETAILED DESCRIPTION

### Definitions

Unless defined otherwise, all technical and scientific terms used herein have the same meaning as commonly understood by one of ordinary skill in the art to which this invention belongs.

“Cannabinoid” as used herein, refers to a class of diverse chemical compounds that interact with cannabinoid receptors (for example, CB1 and CB2) on the cell surface of neurons and other cell types; the term encompassing both cannabis-derived phytocannabinoid compounds and endogenously-produced endocannabinoid compounds, and those synthetically prepared.

“Cannabis”, “Cannabis plant” or “Cannabis spp.” as used herein, refers to any one or more plant(s) from the Cannabis genus of flowering plants in the family Cannabaceae; including but not limited to Cannabis sativa, Cannabis indica and Cannabis ruderalis, and all subspecies thereof (for example, Cannabis sativa subspecies indica including the variants var. indica and var. kafiristanica); including wild or domesticated type Cannabis plants and also variants thereof; including Cannabis plant chemovars (varieties characterized by their chemical composition) which contain different amounts and/or ratios of the individual cannabinoids, terpenes and/or other compounds; including Cannabis plants which are the result of genetic crosses, self-crosses or hybrids thereof; including female and “feminized” plants (which may produce a higher concentration of cannabinoids), and male plants (which may produce a lower concentration of cannabinoids). As is known to the person skilled in the art, Cannabis spp. includes hemp.

“Cannabis plant material” as used herein, refers to plant material derived directly from one or more Cannabis spp. plants; including live or fresh cannabis plants and dried cannabis plants; including but not limited to trichomes, flower buds, flower bracts, leaves, stalk and any other part of cannabis plant.

“Cannabis extract”, as used herein refers to an extract from the cannabis plant, including but not limited to, cannabinoids and terpenes.

“Cannabis suspension” or “cannabis slurry”, as used herein, refers to the partially dissolved suspension of cannabis plant material and solvent that undergoes extraction and/or decarboxylation by the apparatus of the invention.

“Cannabis solution”, as used herein, refers to the fully dissolved solution of cannabinoids extracted from cannabis plant material in solvent that undergoes decarboxylation by the apparatus of the invention.

“Cannabis resin” as used herein, refers to the hydrophobic, viscous, glue-like substance that is produced by extraction (chemical or physical) of various parts of a cannabis plant, in particular glandular trichomes of the flower. Such a resin contains cannabinoids, while reflecting at least some of the molecular diversity of the original cannabis plant, including some or all of terpenes, flavonoids and/or other compounds of interest, some of which may have undergone chemical transformation during the processes used for extraction. Cannabis resin comprises little or no solvent, for example, cannabis resin may comprise between 0-10% solvent, preferably between 0-5%, and most preferably less than 1% solvent by weight.

“Decarboxylated cannabis resin” as used herein, refers to the hydrophobic, viscous, glue-like substance that is produced by extraction (chemical or physical) and decarboxylation of various parts of a cannabis plant, in particular glandular trichomes of the flower. Such a resin contains predominately (>50%, ideally >90%) decarboxylated cannabinoids, while reflecting at least some of the molecular diversity of the original cannabis plant, including some or all of cannabinoids, terpenes, flavonoids and/or other compounds of interest, some of which may have undergone chemical transformation during the processes used for extraction and decarboxylation. The term excludes predominately (>50%) non-decarboxylated resinous substances derived from cannabis (for example, kief, hash, hashish, etc.). Decarboxylated cannabis resin contains little or no solvent, for example, decarboxylated cannabis resin may comprise between 0-10% solvent, preferably between 0-5%, and most preferably less than 1% solvent by weight.

“Decarboxylation” as used herein, refers to a process of removal of a carboxylic group from a cannabinoid molecule such as Δ⁹-THCA or CBDA (an acid form) to the corresponding phenol form such as Δ⁹-THC and CBD; wherein a carboxyl group is removed from the cannabinoid molecule, and carbon dioxide is released.

“Inactive cannabinoid” as used herein, refers to a cannabinoid that has poor potency at the corresponding receptor, often with an EC₅₀ greater than 1 μM; typically a cannabinoid that is in its acidic form such as Δ⁹-THCA, a non-decarboxylated cannabinoid.

### Abbreviations

CB1=cannabinoid receptor type 1, CB2=cannabinoid receptor type 2, CBC=cannabichromene, CBCA=cannabichromenic acid, CBD=cannabidiol, CBDA=cannabidiolic acid, CBG=cannabigerol, CBGA=cannabigerolic acid, CBN=cannabinol, CBNA=cannabinolic acid, CCS=croscarmellose sodium, CMC=carboxymethyl cellulose, DW=deionized water, GC-MS=Gas Chromatography-Mass Spectrometry, HPC=hydroxypropyl cellulose, HPLC=High Performance Liquid Chromatography, ND=Not Detected, NF=National Formulary, PBS=phosphate buffered saline, RDT=rapidly disintegrating tablet, SFE=supercritical fluid extraction, SSG=sodium starch glycolate, MCC=microcrystalline cellulose, THC=tetrahydrocannabinol, THCA=tetrahydrocannabinolic acid, THCV=tetrahydrocannabivarin, THCVA=tetrahydrocannabivarinic acid, USP=United States Pharmacopeia.

### Ranges

As used herein, a range of X to Y includes X and Y and all values in between.

### 1. The Apparatus

An apparatus employing different heating mechanisms such as microwave irradiation technology designed to heat or apply heat to cannabis-carrying slurry/cannabis suspension or cannabis extract in solution for sufficient time including the reaction time is disclosed. The apparatus can withstand the high pressures generated during the heating process, facilitates continuous operation for uninterrupted production, facilitates extraction and decarboxylation of cannabis plant material simultaneously, and accomplishes decarboxylation of cannabis extract, all in a single pass of the cannabis suspension/solution through the apparatus 10. The cannabis suspension/solution can then be subjected to further purification and/or additional processing. For example, the decarboxylated cannabis suspension/solution can be filtered and concentrated to produce a decarboxylated cannabis resin.

In addition to passing a cannabis suspension/solution through the apparatus, it is also possible to solubilize or suspend cannabis resin and pass solubilized or suspended resin through the apparatus for decarboxylation.

Cannabis plant or parts of Cannabis plant serves as a raw material from which phytocannabinoids are to be extracted, and decarboxylated. As is known to those skilled in the art, cannabinoids and cannabinoid-like compounds can also be found in other biological entities, such as Echinacea. Accordingly, the invention is not restricted to cannabis plants, but includes any plants or biological entities that contain cannabinoids.

Decarboxylation is a chemical reaction that releases carbon dioxide and generates neutral cannabinoids. Decarboxylation refers to the conversion of the acid form to the neutral form, whereby a carboxyl group is removed from the cannabinoid molecule, and carbon dioxide is released. For example, in cannabis, the non-psychoactive Δ⁹-THCA can be converted to psychoactive Δ⁹-THC by decarboxylation. Chemical reactions showing decarboxylation of THCA and CBDA are shown below:

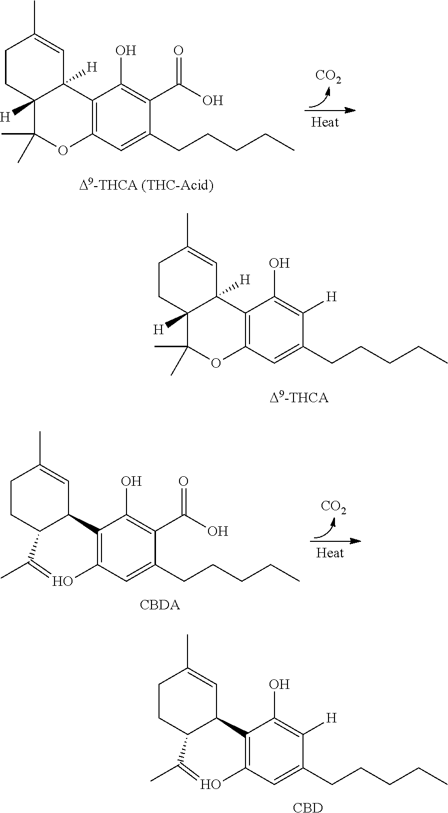

In addition, cannabidiolic acid (CBDA), cannabigerolic acid (CBGA), cannabichromenic acid (CBCA), and tetrahydrocannabivarinic acid (THCVA) may be decarboxylated to yield cannabidiol (CBD), cannabigerol (CBG), cannabichromene (CBC), and tetrahydrocannabivarin (THCV), respectively. In certain embodiments, the cannabinoids in the cannabis resin are at least 55%, 60%, 65%, 70%, 75%, 80%, 85%, 90%, 91%, 92%, 93%, 94%, 95%, 96%, 97%, 98%, 99%, or 100% decarboxylated, (or any integer or fraction percentage values in these ranges, for example, 96.33%) decarboxylated. In some embodiments, THC and/or CBD and/or THCV and/or CBG and/or other major cannabinoids are the major components of the decarboxylated resin, and this is dependent on the particular strain of Cannabis used in the extraction process. In addition to the decarboxylated cannabinoids, other chemicals found in Cannabis spp. and soluble in the solvent used, may be found in the resin. Such compounds may include, for example, terpenes, fatty acids, chlorophyll, flavonoids, and other compounds. Some of the compounds may undergo chemical transformation due to the processes used for extraction and carboxylation.

Plant material can be crushed or ground (milled) into small pieces using any suitable method or device, such as a grinder, pulverizer, blender, and the like. This plant material can be suspended in a solvent that dissolves phytocannabinoids and other chemicals of interest in the Cannabis spp. plants, and the suspension may then be inputted under pressure, such as by use of a pump, through the apparatus, passing through microwave radiation which would heat the suspension to temperatures in the range of 100-200° C., preferably 135-200° C. While apparatus 10 may operate at lower temperatures, such as below 130° C., doing so may require a reduced flow rate of the cannabis input to achieve full decarboxylation. The solvent may comprise an organic solvent, and further, may comprise a pharmaceutically acceptable, reagent-grade, food-grade, or pharmaceutical grade solvent. Alternatively, the plant material can be stirred in a solvent that dissolves the phytocannabinoids and other chemicals of interest, filtered, and the resulting solution can be passed through the apparatus which would heat the solution to temperatures in the range of 135-200° C. Suitable solvents for this purpose could be ethanol, isopropanol, or other similar solvents, among others, and each solvent will require the microwave irradiation to be fine-tuned for maximal heat transfer efficiency. As is known to those skilled in the art, other polar solvents that absorb microwave energy may work, e.g. water, methanol, acetone, acetonitrile, dimethylsulfoxide, N,N-dimethylformamide, methyl ethyl ketone, 1-butanol, 2-butanol, tert-butanol, ethyl acetate, 1-propanol, and various combinations and concentrations thereof.

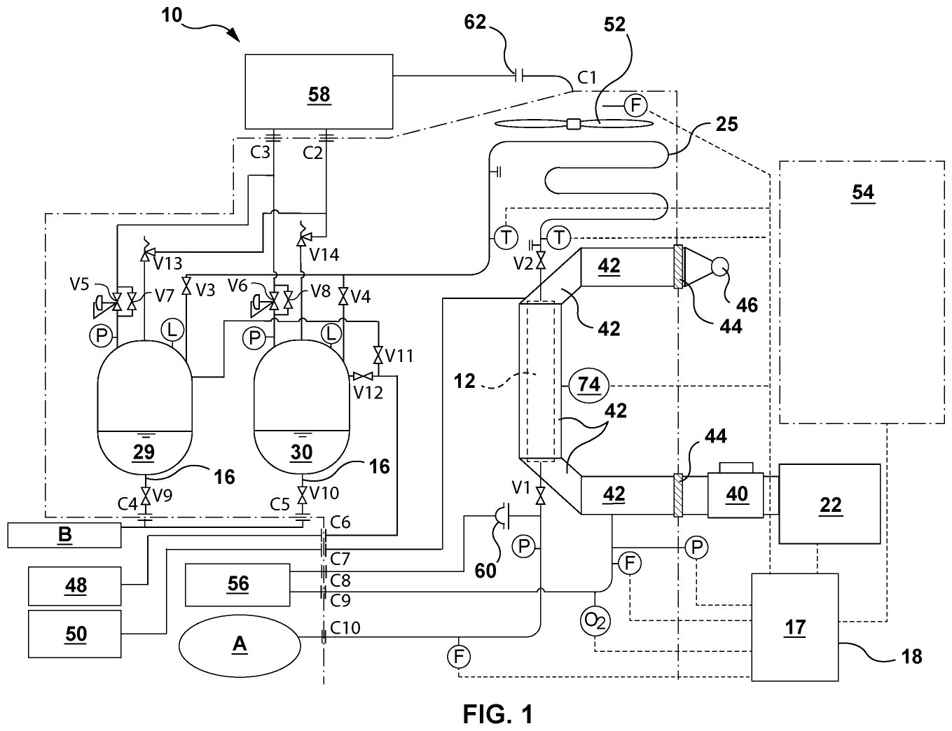

FIG. 1 depicts a schematic diagram of an example embodiment of an apparatus 10 (or Microwave Chemistry Reactor apparatus 10) for continuous input into the apparatus of cannabis solution/suspension/resin, prepared in an organic solvent such as ethanol or isopropanol, and continuous production of a partially or fully decarboxylated cannabis product.

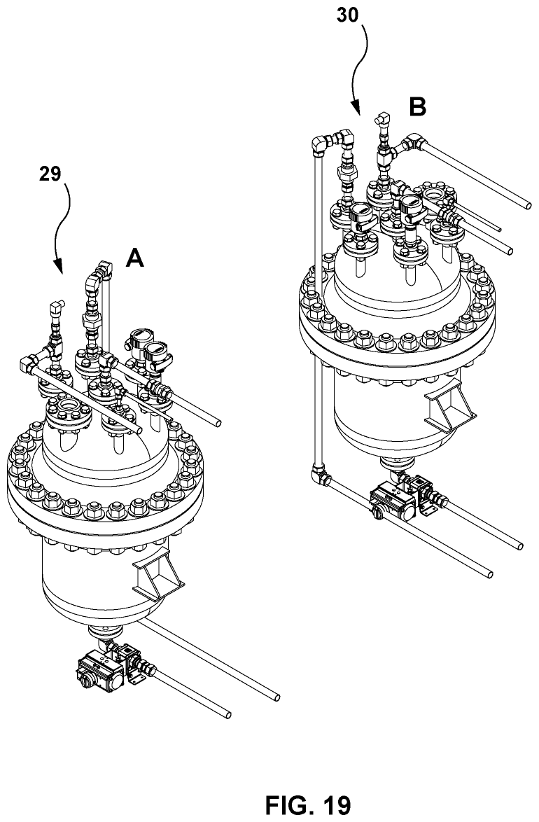

Once heated to the required temperature, high pressure might develop within the reaction vessel or reactor tube(s) 12 because the boiling point of the solvent might be lower than the temperatures 135-180° C. For example, the solvent may comprise a boiling point less than 100° C. Temperature is set depending on the phytocannabinoids content in the plant materials, and the length (time) of exposure required to complete the decarboxylation of the phytocannabinoid acids. Due to high temperature of the suspension and the agitation due to the flow, and optionally, one or more other stirring mechanisms, extraction of the phytocannabinoids into the solvent can also be achieved simultaneously due to their dissolution into the solvent from the crushed plant materials. The stirring mechanism(s) may be incorporated into the apparatus upstream of the reactor tube(s) 12, within the reactor tube(s) 12, downstream of the reactor tube(s) 12 (where stirring continues while the heated slurry passes through the holding tube/pipe 25), and/or may comprise the tube(s) themselves (such as the reactor tube(s) 12). For example, the inner surface of the reactor tube(s) 12 (and/or any of the other piping/tubing of apparatus 10) may comprise inwardly projecting protrusions to facilitate agitation of the cannabis input (e.g., slurry) as it passes through the reactor tube(s). Alternatively, or additionally, the tube(s) (such as the reactor tube(s) 12 and/or holding tube(s)/pipe(s) 25) may also be connected to a device that axially rotates and/or gyrates the tube(s). Other stirring mechanisms of similar or like effect may be used, and it will be appreciated that the flow itself may provide sufficient turbulation. Cannabis spp. plant materials suspension, solution or resin may be subjected to a continuous extraction and decarboxylation process using apparatus 10 as a continuous “flow-through” apparatus by heating the inputted suspension/solution/resin or resin to the required temperature as it passes through one or more reactor tubes 12 using microwave generator 22, and maintaining the temperature for a required time (e.g., as the inputted suspension/solution/resin passes through one or more holding pipes 25 which may be heated, or thermally insulated and subjected to trace heating, to facilitate maintaining the desired temperature) to accomplish complete or near complete decarboxylation, after which the suspension/solution/resin is collected for further processing in one or more receiver vessels 29, 30 (example embodiments of which are shown in FIGS. 19A and 19B, along with portions of connecting tubing/piping). Collection may include cooling the suspension/solution/resin (such as by use of cooling mechanism 31, discussed in greater detail below) to lower temperatures where the solvent is in its liquid state. All such steps are incorporated in such a way that a continuous operation of the apparatus is facilitated permitting industrial scale operations for the production of decarboxylated cannabis suspension/solution/resin, including fully decarboxylated cannabis suspension/solution/resin.

In order to facilitate the above process, in an embodiment, apparatus 10 comprises (i) special design to withstand high temperatures of up to 200° C., (ii) appropriate microwave reactor(s) or generator(s) 22 to elevate the temperature of the cannabis suspension/solution/resin as required, (iii) a special design to withstand resulting high pressures of up to, e.g., 30 bar (although pressure relief mechanisms of apparatus 10, such as those described herein, may be configured to relieve the pressure within the apparatus at pressures below 30 bar, such as at any pressure above 20 bar (it may be required by local regulations that the apparatus be capable of withstanding pressures above (e.g., 1.5×) the operating pressure)), (iv) a special design to incorporate suitable tubing to withstand temperatures and pressures simultaneously permitting flow-through of cannabis suspension/solution/resin in a suitable solvent, and (v) offering a safe environment for operation. Such an apparatus can be charged with a cannabis suspension/solution/resin as an input continuously, or in batches, and the extracted cannabis resin with decarboxylated phytocannabinoids (up to 100% decarboxylated) retrieved as the output continuously or in batches. FIG. 1 depicts a schematic diagram of an example embodiment of an apparatus 10 for continuous input into the apparatus of cannabis suspension/solution/resin prepared in an organic solvent such as ethanol or isopropanol, and continuous production of a partially or fully decarboxylated cannabis product.

FIG. 1 shows a schematic diagram depicting an example embodiment of an apparatus 10 showing the process flow through various components. Input “A” of the cannabis suspension/solution/resin, for example, is charged (such as by application of pressure, described in greater detail below) into apparatus 10 at inlet C10. Inlet C10 may comprise, e.g., a liquid inlet, and may be any suitable diameter. In one embodiment, inlet C10 comprises a liquid inlet. In an embodiment, inlet C10 may comprise a 6 mm diameter. In another embodiment, inlet C10 may comprise, e.g., a 1″ outer diameter and a ⅞″ inner diameter. The cannabis suspension/solution/resin input may then be directed through reactor tube 12 where the cannabis suspension/solution/resin is exposed to appropriately tuned microwave radiation via microwave generator 22 to elevate the temperature of the suspension/solution/resin. Alternate or conventional heating techniques, such as flame heating, may also or alternatively be employed. It is expected that the length of reactor tube 12 is sufficiently long (e.g., about 30 cm to about 200 cm), with an appropriate internal or interior diameter (e.g., about 4 mm to about 120 mm), to allow for sufficient exposure of the cannabis suspension/solution/resin input to the heating mechanism (e.g., microwave irradiation). It will be appreciated that more than one heating mechanism could be used, of the same or of different types, for heating the reactor tube(s) 12 and/or for applying heat downstream of the reactor tube(s), as described further below. Valve V1 may be arranged under (where reactor tube(s) 12 is vertically arranged) or downstream of reactor tube(s) 12 to allow for reactor tube 12 isolation. The internal diameter of the tubing used in the apparatus will define the volume of the cannabis suspension/solution/resin or input that can flow through the apparatus per unit time. It is expected that the inputted suspension/solution/resin may continue to move through the piping or tubing of apparatus 10 allowing for the continuous charging of or supply of cannabis suspension/solution/resin to the apparatus.

Irradiation of the inputted cannabis suspension/solution/resin occurs during the time the suspension/solution/resin spends in reactor tube 12, which may be formed from quartz, or other suitable materials, such as steel or brass. The heat and pressure generated in reactor tube(s) 12 is retained in piping 25 (e.g., holding pipe(s) 25, shown in isolation in FIG. 13) connected to the exit of the reactor tube(s), which may comprise stainless steel tubing, for example. Holding pipe(s) 25 may be connected to and contiguous with reactor tube(s) 12 outlet. Holding pipe(s) 25 may be long enough to allow for complete decarboxylation of phytocannabinoids at a set temperature, and withstand the high pressure generated due to the heating of the solvent. For example, holding pipe 25 may be several meters long, may be heated directly, or insulated and subjected to trace heating, by one or more other heating mechanisms (e.g., flame, thermal or infrared radiation), to maintain the set temperature achieved within reactor tube 12, and is exited into one or more receiver vessels, each of appropriate volume depending on the parameters of production (as examples only, the receiver vessels may be capable of holding 10 L, 20 L, or higher of cannabis product) allowing for cooling and subsequent collection for further processing. As further described below, apparatus 10 may comprise cooling mechanism 31 for cooling of the cannabis suspension/solution/resin prior to collection in the receiver vessel(s).

Collection of the processed (decarboxylated) cannabis suspension/solution/resin may occur alternately, such that the cannabis suspension/solution/resin flows at high pressure through the apparatus tubing and into one receiver vessel 29 or 30 at a time. Once the collecting receiver vessel is almost full, it can be cooled (or further cooled, where a cooling loop is employed prior to collection in the receiver vessels) and emptied through output(s) or output port(s) 16 for further processing of the collected decarboxylated cannabis suspension/solution/resin, but the high-pressure flow can continue, as the cannabis suspension/solution/resin can then be collected in the available receiver vessel, thus permitting a continuous operation of the apparatus at the set high temperature, and high pressures. Valves V9, V10 may be disposed downstream of the outlets 16 (which may comprise liquid outlets 16) of receiver vessels 29, 30 to selectively block or open the path from outlets 16. This mode of operation provides the ability to extract and decarboxylate cannabis plant materials in large scales, for example several kilograms or several tens of kilograms per hour, depending on the diameter of the tubing of the apparatus, length of the tubing (or piping), flow rate at which the input is charged into the apparatus, the number and type of heating mechanisms used to heat and maintain the temperatures, the number of reactor tubes utilized, and the number of receiver vessels. Flow of the suspension/solution/resin between various components of the apparatus, including into the one or more, preferably two or more (to allow for alternate collection and to facilitate continuous flow of the suspension/solution/resin) receiver vessels 29, 30, may be controlled by one or more valves (shown in various positions in FIG. 1 with the prefix “V”). Connections, or inlets/outlets/ports are also shown in various positions in FIG. 1 with the prefix “C”. FIG. 1 depicts one possible embodiment of the distribution of valves and connections/inlets/outlets in an example apparatus, and Table 1, below, provides a legend for the example valves/connections/inlets/outlets shown in FIG. 1, along with example configurations thereof in the one example embodiment.

TABLE 1
Example Connections and Valves

| Connection/ | |
|---|---|
| port (C) or | |
| Valve (V) No. | Description |
| C1 | Extraction (e.g., 402 mm × 402 mm) to safe |
| | venting location 58 |
| C2 | Pressure relief (e.g., 12 mm) to safe venting |
| | location 58 |
| C3 | Gas discharge (e.g., 12 mm) to safe venting |
| | location 58 |
| C4 | Vessel 1 liquid discharge (e.g., 12 mm) |
| C5 | Vessel 2 liquid discharge (e.g., 12 mm) |
| C6 | High pressure nitrogen supply (e.g., 12 mm) |
| C7 | Low pressure nitrogen supply (e.g., ⅛″) |
| C8 | Liquid pressure relief (e.g., 12 mm) |
| C9 | Waveguide vent/drain (e.g., 8 mm) to safe |
| | venting location/drain 56 |
| C10 | Liquid inlet (e.g., 6 mm) |
| V1 | Reactor isolation (e.g., bottom) |
| V2 | Reactor isolation (e.g., top) |
| V3 | Vessel 1 product inlet or input port |
| V4 | Vessel 2 product inlet or input port |
| V5 | Vessel 1 back pressure regulator |
| V6 | Vessel 2 back pressure regulator |
| V7 | Vessel 1 regulator by-pass |
| V8 | Vessel 2 regulator by-pass |
| V9 | Vessel 1 product discharge or output (e.g., |
| | liquid outlet) |
| V10 | Vessel 2 product discharge or output (e.g., |
| | liquid outlet) |
| V11 | Vessel 1 nitrogen feed/supply |
| V12 | Vessel 2 nitrogen feed/supply |
| V13 | Vessel 1 safety relief valve (for pressure |
| | relief) |
| V14 | Vessel 2 safety relief valve (for pressure |
| | relief) |

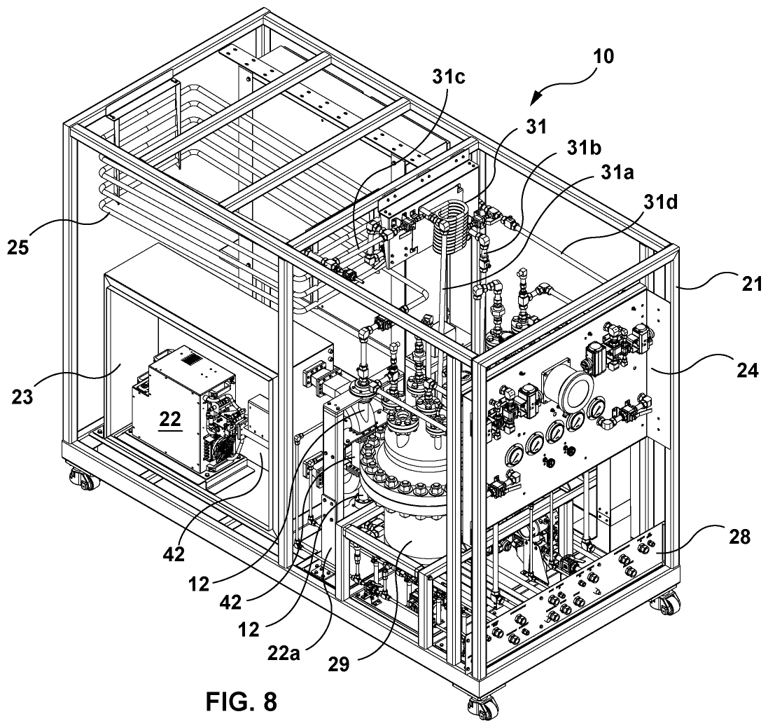

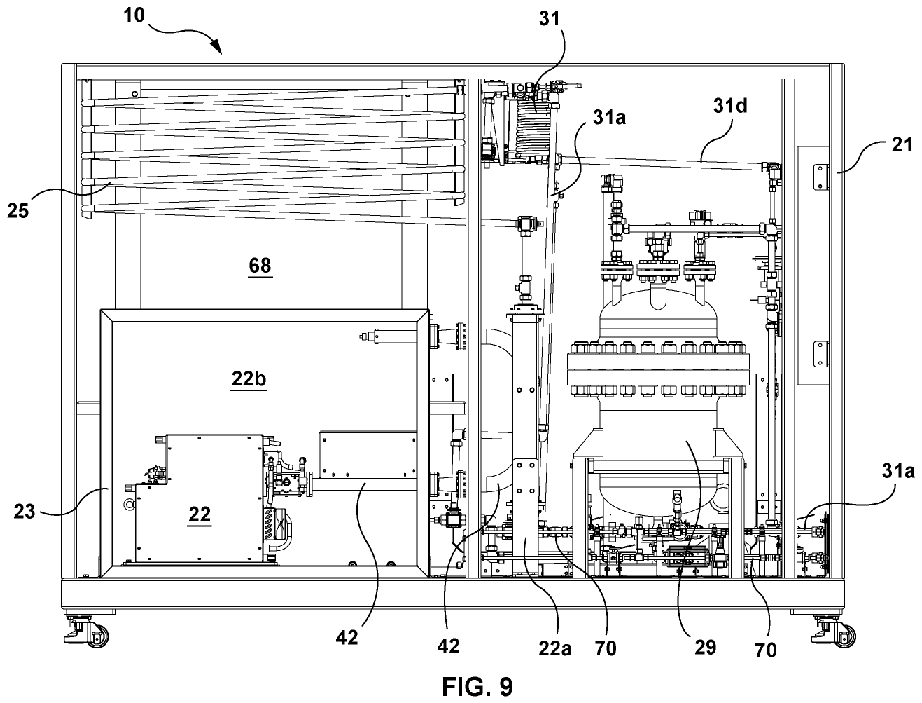

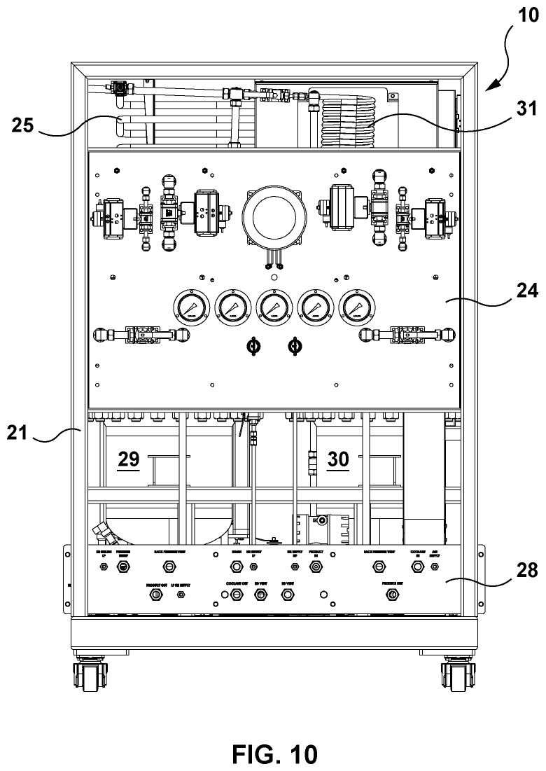

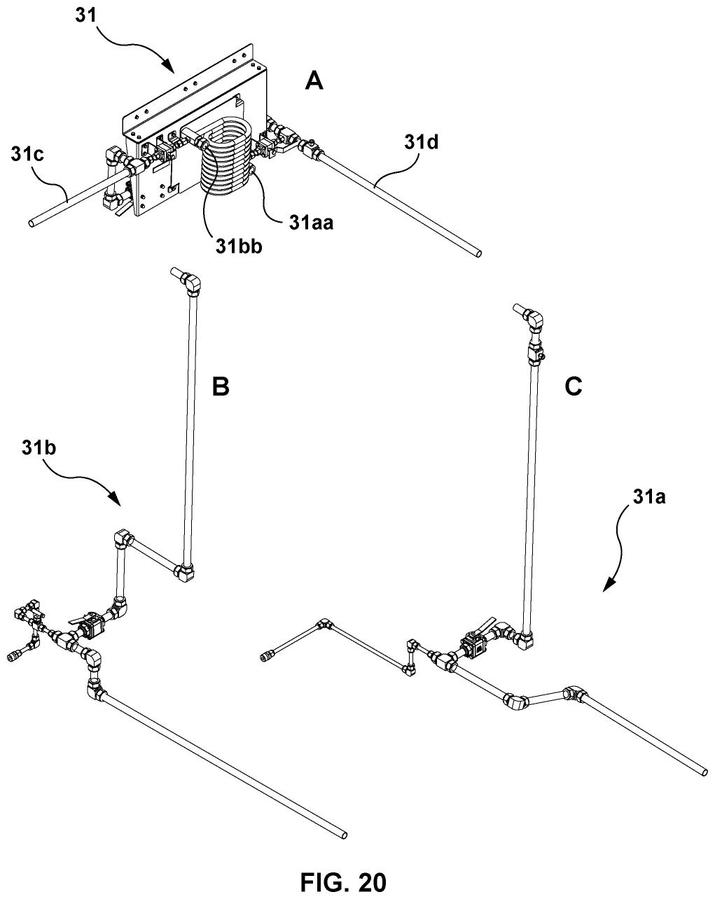

With reference to FIGS. 8, 9, 10, and 20A, apparatus 10 may include cooling mechanism 31 to cool the suspension or solution. For example, a cooling loop/coil (also called a heat exchanger) 31 may be used cool the effluent from holding pipe 25 to, for example, less than 45° C., less than 50° C., or less than 60° C. (depending on operating parameters and the solvent used for the cannabis suspension/solution/resin) as the suspension/solution/resin exits the holding pipe 25 prior to filling the receiver vessel 29, 30. With reference to FIGS. 8 and 20, apparatus 10 may comprise intermediate tubing/piping 31c for carrying the cannabis suspension/solution/resin/slurry from holding pipe 25 to cooling loop/coil/pipe 31, and tube/pipe 31c may pass within and through cooling loop/coil/pipe 31, so that cooling loop 31 comprises an inner diameter larger than the outer diameter of the tubing/piping 31c carrying the cannabis suspension/solution/resin/slurry through the cooling mechanism. Cooling loop/pipe/coil 31 may be fed with a cooling solution for cooling the cannabis suspension/solution/resin/slurry as it flows through the cooling mechanism. The inner diameter of cooling loop/coil/pipe 31 may be dimensioned sufficiently larger than the outer diameter of tube 31c to carry within and around tube 31c a sufficient amount of cooling fluid to effect the desired amount of cooling within the length of cooling loop 31. It will be appreciated that the length and inner/outer diameters of cooling loop 31 may be adjusted as required for effecting the desired cooling. With reference to FIGS. 8 and 9, the cooled cannabis suspension/solution/resin/slurry may flow from cooling mechanism 31 to the receiver vessel(s) via exit tube/pipe 31d leading from the cooling mechanism to the receiver vessel(s). In another embodiment, the cooling mechanism may alternatively, or additionally, comprise a means for cooling the receiver vessel(s) themselves, such as by use of cooling jacket(s) (not shown) disposed on or about all, some, or substantially all of the receiver vessel exterior surface(s). Where required by the cooling mechanism, the cooling mechanisms described herein may employ any suitable substances, such as water, glycol or Freon, flowed through the cooling mechanism by conventional means (e.g., using a pump), for effecting cooling. It will be appreciated that yet other cooling mechanisms 31 may be used, or that cooling loop 31 shown in FIG. 8 may effect cooling other than by passage of the cannabis suspension/solution/resin/slurry within it (e.g., tubing 31c may be arranged to pass adjacent to and in contact with a cooling mechanism, such as cooling loop 31, such that the cooling mechanism draws heat from tube 31c and its contents).

Apparatus 10 may comprise a radar level detector and a temperature sensor to monitor the temperature of the suspension/solution/resin/slurry as it empties into the receiver vessels.

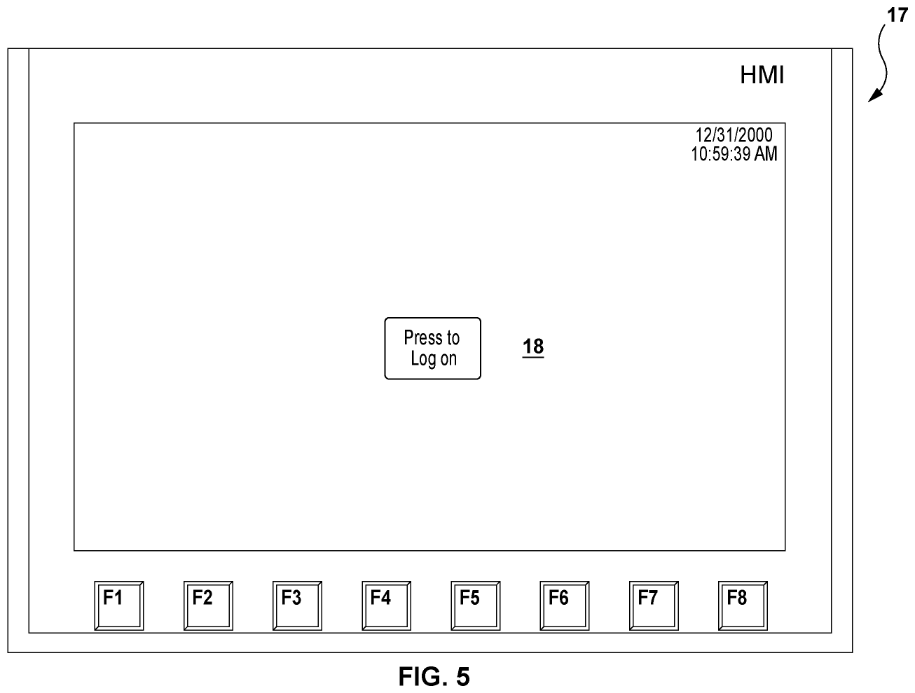

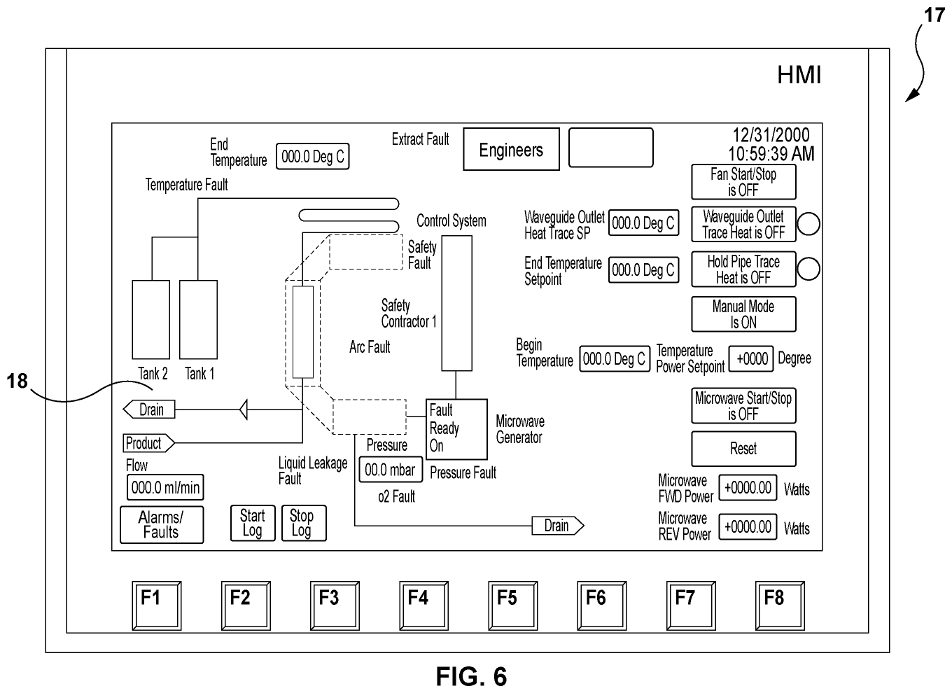

With reference to FIGS. 1, 5 and 6, apparatus 10 may be connected to or comprise a control panel/Programmable Logic Controller (PLC) 17 that may include a Human Machine Interface (HMI) 18 to allow for the control of and monitoring of various aspects of the apparatus. Sensor(s) may be distributed at various points of the apparatus which may be connected to the controller/PLC 17 to provide sensor readings to HMI 18 to allow efficient monitoring of apparatus parameters, such as pressure, temperature and flow, and adjustment thereto. Gauges (such as pressure or temperature gauges, or flow meters) may also, or alternatively, be included at various points of the apparatus to allow for monitoring of pressure and temperature or other settings. The HMI may comprise a touch screen or other form of input device capable of displaying or communicating sensor readings from sensor(s) of apparatus 10. The example HMI shown in FIG. 5 and FIG. 6 is discussed in greater detail, below.

The apparatus may be designed for continuous flow operation and/or batch operation. The solvent may include potentially flammable organic solvents such as ethanol, and as such the apparatus is designed to account for this hazard. The apparatus may receive an ethanol, isopropanol or water based slurry containing plant material. The apparatus may be designed to comply with government regulations in various jurisdictions for under pressure vessels. For example, the apparatus may be configured to operate in a Class 1 Division 2 hazardous environment (as classified according to the hazardous location classification system under the National Fire Protection Association (NFPA) Publication 70, National Electric Code® (NEC) (similar codes and classifications may apply in other jurisdictions, such as the Canadian Electrical Code in Canada or the ATEX directive in the European Union)) by encasing the electrical components of the instrument in a nitrogen pressurized cabinet and using intrinsically safe wiring. Nitrogen may be supplied to the apparatus such as by C6, C7, V11, and V12, as shown in the specific example embodiment of FIG. 1 and Table 1.

In the embodiment shown in FIG. 1, typical operation pressure may be approximately 10-20 bar with a temperature of up to 180° C. The example apparatus shown in FIG. 1 has a maximum working temperature of 180° C. and pressure of 20 bar (290 psi), but in other embodiments, the apparatus may withstand temperatures up to 190° C. or 200° C., and higher pressures (e.g., up to 30 bar).

With reference to FIG. 1, apparatus 10 may further comprise a stub tuner 40, a waveguide 42 to carry and guide the microwaves generated by microwave generator 22, one or more windows 44 into the waveguide, sliding short 46, high pressure nitrogen supply 48, low pressure nitrogen supply 50, extraction fan 52, and electrical cabinet 54. The nitrogen may be used to pressurize the apparatus in addition to maintaining an inert environment (for the purposes of reducing the potential for hazardous electrical events). For example, low pressure nitrogen supply 50 may be used to help maintain an inert environment around the electrical components of apparatus 10, and may further be used to purge contents of apparatus 10. Low pressure nitrogen supply 50 may be provided to supply an inert protective gas as a safety feature to reduce the chance of spark formation in the event that, e.g., reactor tube 12 (which may comprise a quartz tube, for example) cracks. High pressure nitrogen supply 48 may be used to pressurize apparatus 10, and may also be used to purge contents of apparatus 10. In an embodiment, the high pressure nitrogen feed 48 may purge the piping system and receiver vessels of apparatus 10, while the low pressure nitrogen feed 50 may purge the wave guide 42 and electrical cabinets of apparatus 10. Apparatus 10 may further comprise a third nitrogen supply (not shown) for supplying nitrogen at low pressure into electrical cabinet 54 of apparatus 10, to further facilitate maintenance of a spark-proof environment, or an environment at low risk of electrical spark. The electrical cabinet may be sealed and pressurized. To address any loss of pressure in the electrical cabinet, which may occur slowly over time, the low pressure input of nitrogen is expected to maintain the environment of the electrical cabinet at a positive pressure of at least 25 Pa, and electrically inert. The electrical cabinet may comprise pressure sensor(s) communicatively coupled to the controller/PLC 17, and the controller/PLC may be programmed to shut down apparatus 10 in the event that pressure within the electrical cabinet falls to below a safe operating pressure. Electrical cabinet 54 may comprise, in the specific example embodiment of FIG. 1, the following controller interfaces (as an example only): digital outputs; volt-free contacts for microwave general fault; a microwave “on” function; a microwave “ready” indicator; trip temperature; trip arc; liquid leakage indicator (communicatively coupled to one or more liquid sensors arranged at suitable locations along the apparatus tubing/piping); an “extraction off” function; trip inert environment; a “no flow” indicator; analogue outputs (e.g., 4-20 mA or 0-10V for temperature and microwave power); and digital input (e.g., 2 off volt-free safety contacts).

Apparatus 10 may employ electricity to open/close the valves described herein or, alternatively, apparatus 10 may employ compressed air to open/close the valves described herein. The use of compressed air for valve actuation, rather than electricity, is expected to provide for a safer operating environment. The valves described herein may also be actuated depending on readings from level, temperature and/or pressure sensors. The controller/PLC 17 may actuate the valves based on the sensor readings. Any of the valves described herein may also be manually actuated. Apparatus 10 may comprise any of a number of suitable sensors for monitoring aspects of the operation of the apparatus. For example, as shown in the specific example embodiment of FIG. 1, apparatus 10 may comprise flow meter F, pressure gauge P, oxygen sensor O₂, temperature probe T, and liquid level sensor L. It will be appreciated that other types of sensors may be utilized, and multiples of each type may be utilized for redundancy and/or greater accuracy via processing of multiple sensor readings by controller 17.

The apparatus shown in FIG. 1 may produce high levels of non-ionizing microwave radiation. The potential for dangerous levels of exposure are mitigated by safety protocols and tunability of the equipment. This microwave irradiation is generated by an appropriate microwave generator 22 and serves as a heating mechanism of the input cannabis suspension/solution/resin. Microwave irradiation is guided via a microwave guide 42. Apparatus 10 may also comprise arc detector 74 for detecting arc events and breaking the circuit in order to protect the apparatus from electrical fires. Cannabis suspension/solution/resin, after entering the apparatus as input, flows through reactor tube(s) 12, and is exposed therein to microwave irradiation which is guided to the reactor tube via waveguide 42. This process heats the input to the set temperature, and if this temperature is higher than the boiling point of the solvent in the input, then higher pressures will be developed in the apparatus. The apparatus can be pressurized to 140-150 psi (˜10 bar) prior to operation. Generally, pressure within apparatus 10 may increase over the course of operation, subjecting the input to a back pressure and necessitating higher pressures to flow the input through the apparatus. Reactor tube 12 may comprise quartz if the heating mechanism is microwave irradiation, or could be formed from stainless steel, brass or other non-reactive metal conductor (e.g., where the heating mechanism comprises a flame).

Apparatus 10 may comprise safety systems such as a rupture disc system 60. The rupture disc maybe configured to rupture at any suitable pressure, depending on operating parameters of the apparatus. For example, in the specific example apparatus shown in FIG. 1, rupture disc 60 may have a maximum pressure setting of 23.5 bar (˜340 psi) at which point it will rupture and vent any volatile or other materials to safe location/drain 56. During operation, the venting area(s) should not be blocked or obstructed at any time. Another venting mechanism is shown in FIG. 1 through C1, which is an extraction connection 62 for venting vapours and other volatile materials. It will be appreciated that such extraction connections/ports need not be limited to the specific locations shown in the drawings. An area surrounding apparatus 10 may be zoned according to local regulations for dangerous (e.g. explosive) substances in the atmosphere.

It will be understood by the skilled person that apparatus 10 would be for operation only by a designated individual that has completed appropriate operating and safety training for apparatus 10.

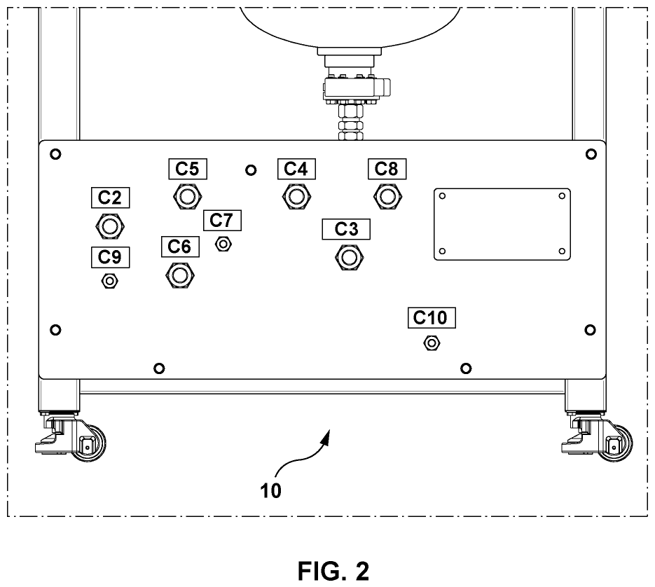

FIG. 2 illustrates an example configuration for connections in one example embodiment of the Microwave Chemistry Reactor apparatus 10, which may assist in achieving the continuous flow of cannabis suspension/solution/resin input into the apparatus. These connections/ports are as described in Table 1 and as shown in FIG. 1, and FIG. 2 depicts one example arrangement of the connections/ports on a side of apparatus 10, such as for connection of apparatus 10 to a pressure relief area or a safe venting location which may, e.g., be located outside of the building or facility housing apparatus 10. The connections shown in FIG. 2 include, as described above: C10 for the input of the cannabis slurry/cannabis solution in the organic solvent; C6 and C7 for input of an inert gas, such as nitrogen, to either maintain the inert atmosphere in the apparatus (at low pressure), or maintain the inert atmosphere in the flow pipes and the receiver vessels (at high pressure) and other areas to replace air/oxygen, limiting the potential for explosion or fire; C4 and C5 for output of the cannabis product from the receiver vessels 29, 30 to yield output “B”; and C2, C3, C8, and C9 for pressure relief/drain connections to discharge areas.

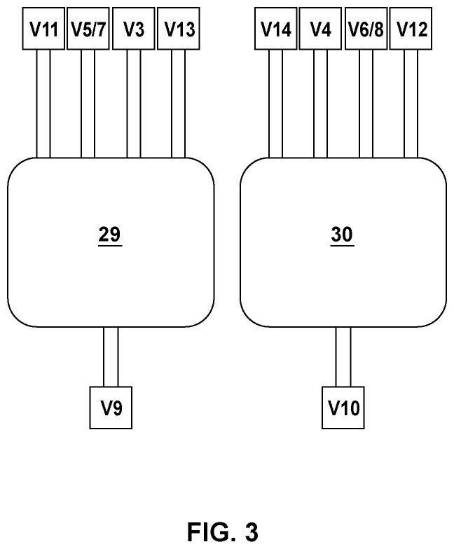

FIG. 3 shows an example embodiment of various valves in the embodiment of apparatus 10 shown in FIG. 1 and as described in Table 1, with two receiver vessels 29, 30 permitting an alternate collection process of the decarboxylated/extracted cannabis suspension/solution/resin. Optionally, apparatus 10 may comprise a radar level detector and a temperature sensor to monitor the temperature of the product as it empties into the vessel.

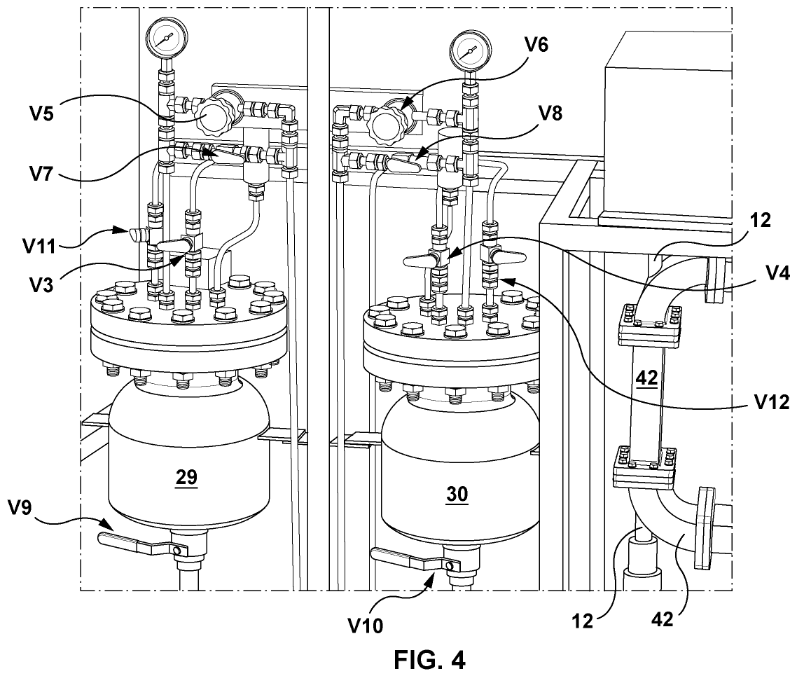

FIG. 4 shows a partial front view of the embodiment of apparatus 10 illustrated in FIG. 1, showing various components including receiving vessels 29, 30, microwave guide 42, reactor tube 12, and various valves V3 to V12.

As discussed above, a Human Machine Interface (HMI) may be used to control various components of apparatus 10, such as various connectors and valves, and the microwave generator 22, and monitor the flow of the cannabis suspension/solution/resin and control any of the functions of apparatus 10 as well as of equipment connected to apparatus 10. FIG. 5 depicts an example Logon screen of an embodiment of an example HMI. FIG. 6 depicts an example HMI, illustrating various digital controllers and sensor readouts. The example HMI of FIG. 6 comprises various readouts from several apparatus components, including microwave irradiation parameters, temperature and pressure readings, the flow rate of the input, and other controls and readouts, as shown, all of which can be monitored or controlled via HMI 18.

Apparatus 10 is expected to have value in an industrial setting to extract and decarboxylate cannabis to obtain cannabis resin in a consistent manner and in a large scale, e.g., in multi-kilograms scale due to its continuous operation capability. For example, in the example embodiment shown in FIG. 8, production of a decarboxylated cannabis product is expected to be in the range of ˜1-2 kg/hr, and higher yields may be achieved by adjusting parameters of apparatus 10, e.g., the diameter of the tubing of the apparatus, length of the tubing (or piping), flow rate at which the input is charged into the apparatus, the number and type of heating mechanisms used to heat and maintain the temperatures, the number of reactor tubes used (described below), and the number and size of the receiver vessels.

Typically, for efficient heating of the input to apparatus 10, the inner diameter of reactor tube 12 will need to be limited in order for the heating mechanism (whether, e.g., microwave irradiation or flame) to effectively heat the cannabis suspension/solution/resin throughout to a generally consistent temperature. Larger diameter tubing could potentially result in hotter spots in the cannabis suspension/solution/resin closer to the heat source (e.g., closer to the walls of the tubing) and colder spots at the interior portions of the cannabis suspension/solution/resin. As such, it may be desirable to maintain the diameter of reactor tube 12 to no more than 15-20 mm. For example, in the example apparatus shown in FIG. 1, the inner diameter of the reactor tube is 6 mm. In the example embodiment shown in FIG. 8, the reactor tube is a quartz reactor tube with an outer diameter of 40 mm and an inner diameter of 20 mm. It will be appreciated that other dimensions for the reactor tube(s) 12 may be utilized, and that larger diameter reactor tubes may suffice depending on whether agitation or stirring mechanisms are employed to facilitate even distribution of heat throughout the cannabis input, or if the flow rate itself results in sufficient turbulation of the cannabis input to sufficiently distribute the heat throughout the input.

In another example embodiment of apparatus 10, to more efficiently heat the input and/or to increase the volume of cannabis suspension/solution/resin that can be input to the apparatus per unit time, the input is divided into multiple reactor tubes 12 for heating to the desired temperature by a heating mechanism (such as microwave irradiation or flames). In this embodiment, where the multiple reactor tubes 12 employ smaller diameter tubing, the heating may be effected in a more efficient and effective manner, to achieve more consistent heating throughout the cannabis suspension/solution/resin. Additionally, or alternatively, multiple reactor tubes 12 may be used to increase throughput of the apparatus, by employing larger diameter tubing into and out of the reactor tubes, and accordingly increasing the number of reactor tubes to accommodate the greater rate of input to the apparatus and thus to the reactor tubes. In this scenario, each reactor tube may or may not be of a smaller diameter; where each reactor tube is of a smaller diameter, it may be possible to achieve both greater throughput and more efficient and effective heating of the cannabis suspension/solution/resin.

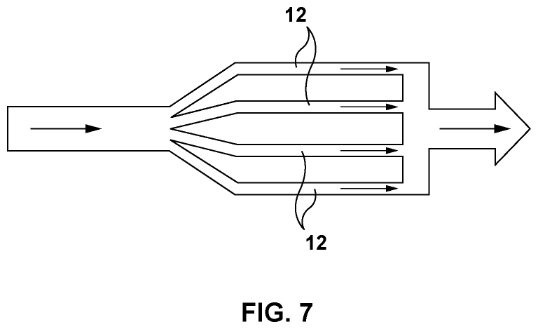

FIG. 7 illustrates the conceptual arrangement of the tubing in an embodiment of apparatus 10 comprising multiple reactor tubes 12, to achieve the heating of the input efficiently (while potentially also increasing throughput, where the multiple reactor tubes accommodate larger diameter tubing at the inputs and outputs to/from the reactor tubes 12, as discussed above). The cannabis suspension/solution/resin flows in the direction of the arrows shown in FIG. 7, from the reactor tubes 12 and onward to holding pipe 25 of desired length where the desired set temperature reached in the reactor tubes 12 will be maintained. An embodiment of apparatus 10 with a single reactor tube 12 may be upgraded to replace the reactor tube with a multiple reactor tubes 12 (as shown in FIG. 7), to effect more efficient and effective heating of the cannabis suspension/solution/resin (where each individual reactor tube comprises a smaller inner diameter than the replaced reactor tube) and/or to allow for increased throughput of the cannabis suspension/solution/resin to/from the reactor tubes 12.

As described above, FIG. 7 illustrates a conceptual example of an apparatus 10 comprising multiple reactor tubes 12 to divide input (such as cannabis slurry) into multiple, and potentially smaller tubes for efficient heating and/or greater throughput, and operating in a continuous flow manner. Due to the need for larger inner diameter tubes (e.g., 20 mm, 30 mm or higher) to facilitate larger volumes of slurry to flow through, for efficient heating, the input from a larger inner diameter pipe may be divided into two or more smaller inner diameter reactor tubes 12 (each with an inner diameter of, e.g., 6-20 mm) of appropriate length, which then converge into a larger inner diameter tube (which may comprise, e.g., holding pipe 25 (as described above). The holding pipe is configured to be of a set length that permits sufficient time for decarboxylation and extraction to take place while the cannabis suspension/solution/resin flows therethrough, and the decarboxylated cannabis product is ultimately directed into receiver vessel(s) for collection (after passing through cooling mechanism 31, where employed).

A further example embodiment of apparatus 10 is shown in FIGS. 8, 9 and 10, showing frame assembly 21, microwave generator 22, microwave assembly 22a, enclosure assembly 23, upper manifold 24, holding pipe 25, lower bulkhead plate 28, receiver vessels 29, 30, cooling mechanism 31, waveguide 42, and reactor tube 12. As shown in FIG. 8, reactor tube(s) 12 may enter waveguide 42 at one point, pass through waveguide 42, and exit waveguide 42 at another point. In the embodiment shown in FIG. 8, the waveguide is arranged such that it extends horizontally from microwave generator 22, and then bends vertically upward. In this embodiment, and as also shown in FIG. 15, reactor tube 12 enters the waveguide toward the bottom of the vertical segment of the waveguide at 42a, and exits the waveguide toward the top of the vertical segment of the waveguide at 42b. It will be appreciated that other configurations or arrangements of waveguide 42 and reactor tube(s) 12 may be employed, provided that a sufficiently long segment of the reactor tube(s) 12 pass through the waveguide 42 for exposure to the microwave irradiation to effect the desired degree of decarboxylation. It will be further appreciated that a waveguide may also be used in embodiments employing infrared radiation as a heating mechanism, and further, that waveguides may not be required in some embodiments (such as where flame heating is utilized to initially heat the cannabis suspension/solution/resin in the reactor tube(s) 12).

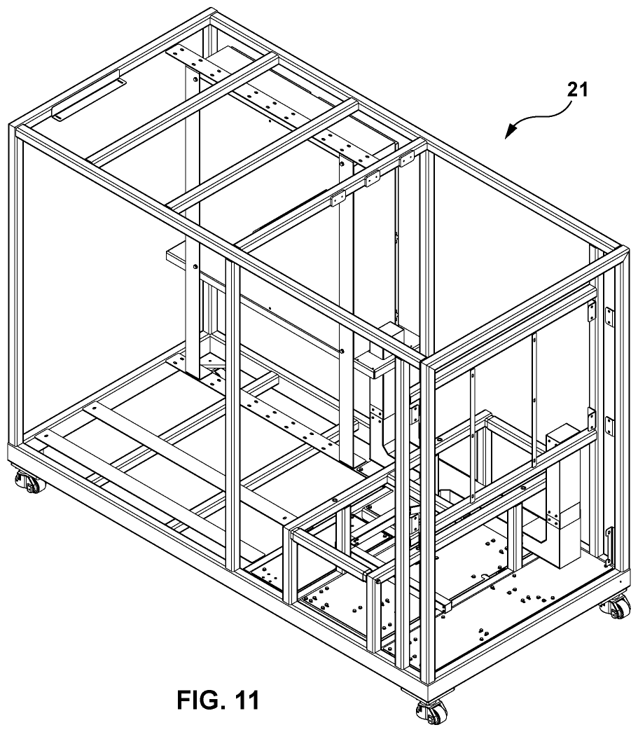

Frame assembly 21 comprises the frame and support for the components of apparatus 10, and may be of any suitable dimensions to accommodate the apparatus components, which component dimensions will depend upon the desired parameters of apparatus 10. In the specific example embodiment shown, frame assembly 21 is 3.06 m×1.47 m×2.28 m. The specific example embodiment of frame assembly 21 shown in FIG. 8 is shown in isolation in FIG. 11.

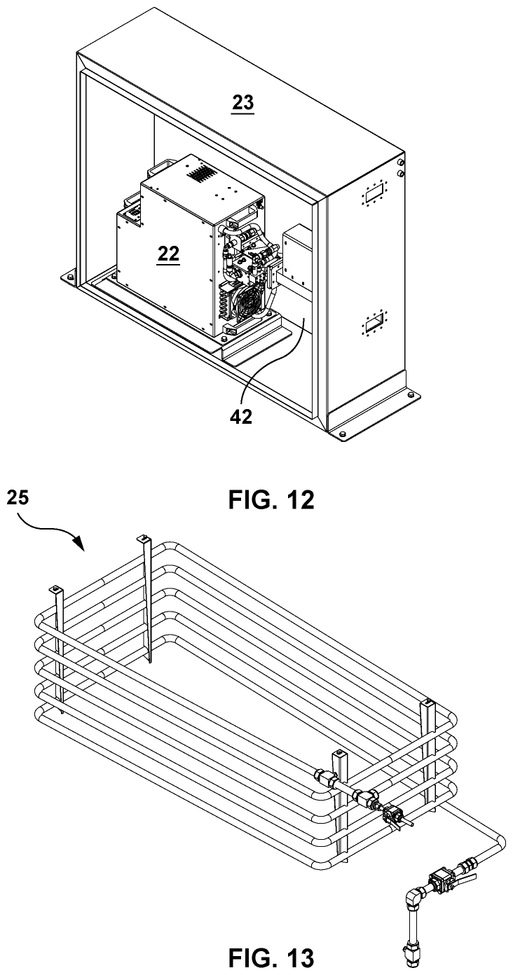

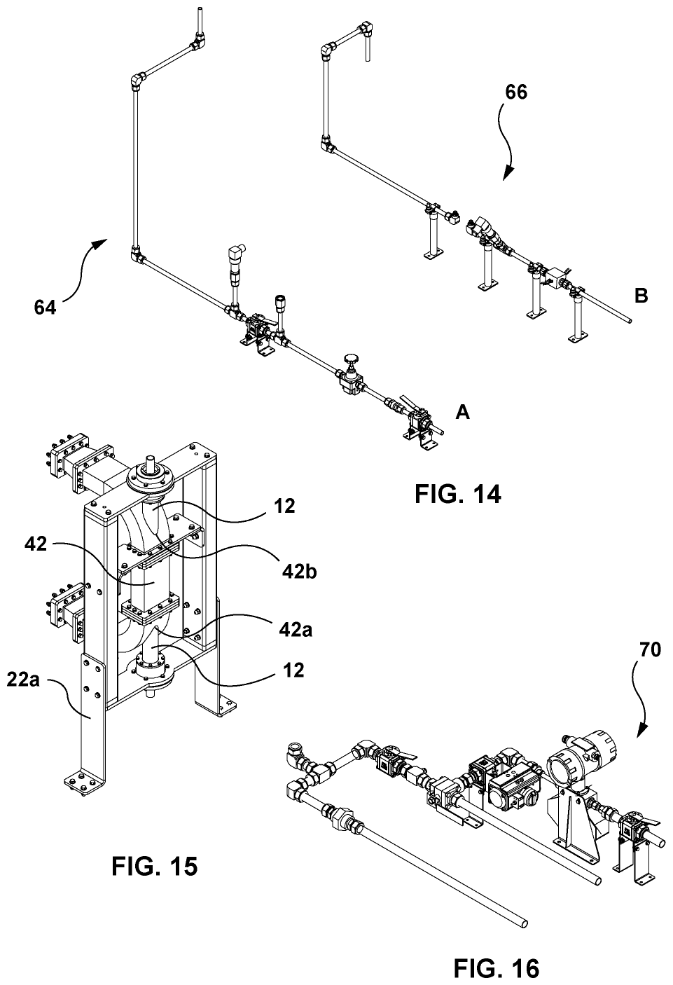

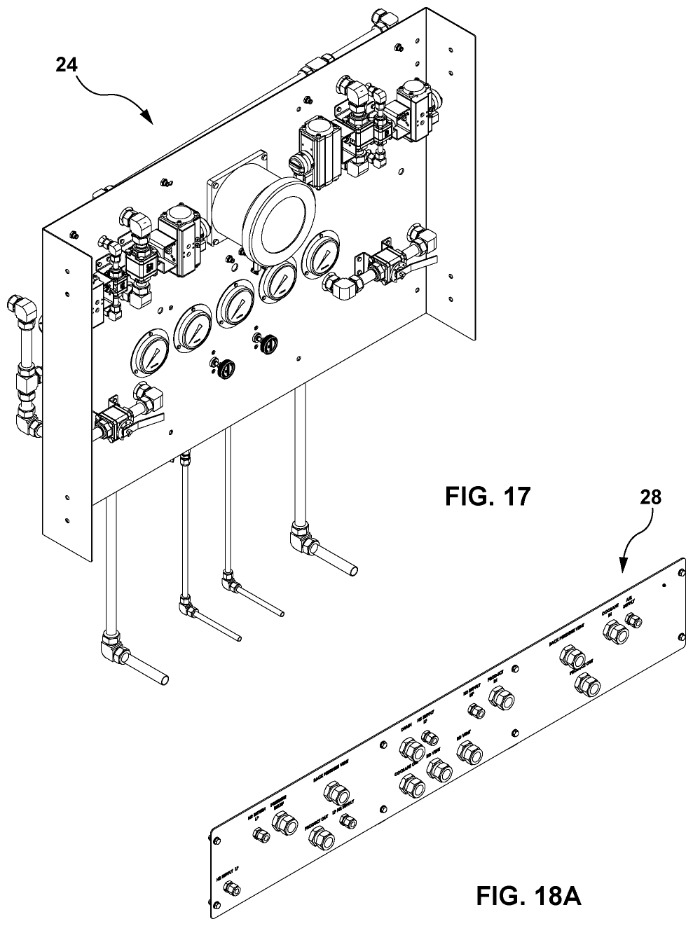

Microwave assembly 22a provides the frame to support microwave generator 22 and waveguide 42. The specific example embodiment of microwave assembly 22a shown in FIG. 8 is also shown in FIG. 15, along with reactor tube 12 and portions of waveguide 42. Apparatus 10 may also comprise enclosure assembly 23 (shown also in FIG. 12), which provides a cabinet housing for the microwave generator 22 to form a microwave cabinet 22b (see FIG. 9). Microwave cabinet 22b may be purged with nitrogen via nitrogen feed assembly 64, shown in isolation in FIG. 14A. Apparatus 10 may also comprise a control cabinet 68 for housing electrical controls and components, the rear of which is shown in FIG. 9, such that nitrogen supplied to the microwave cabinet 22b via nitrogen feed assembly 64 may then flow into control cabinet 68 to thereby purge and render inert the environment within control cabinet 68, to thereby reduce the risk of hazardous electrical events. The cabinets may slowly discharge nitrogen into the surrounding environment, and apparatus 10 may comprise one or more sensors for determining when the pressure within the cabinets (e.g., cabinet 68) drops to below a predetermined threshold level (e.g., 30 Pa), to then communicate same automatically to controller 17 for the controller to trigger actuation of an actuator valve to supply further nitrogen to the cabinets. Apparatus 10 may also comprise a nitrogen exhaust assembly 66 (shown in isolation in FIG. 14B), for the exhaust of nitrogen from within waveguide 42. Further, apparatus 10 may comprise the upper manifold 24, shown along with some connecting pipes in FIG. 17. Upper manifold 24 provides a housing for the various pressure gauges and the actuator valve assembly of apparatus 10, as shown in FIGS. 8, 10 and 17.

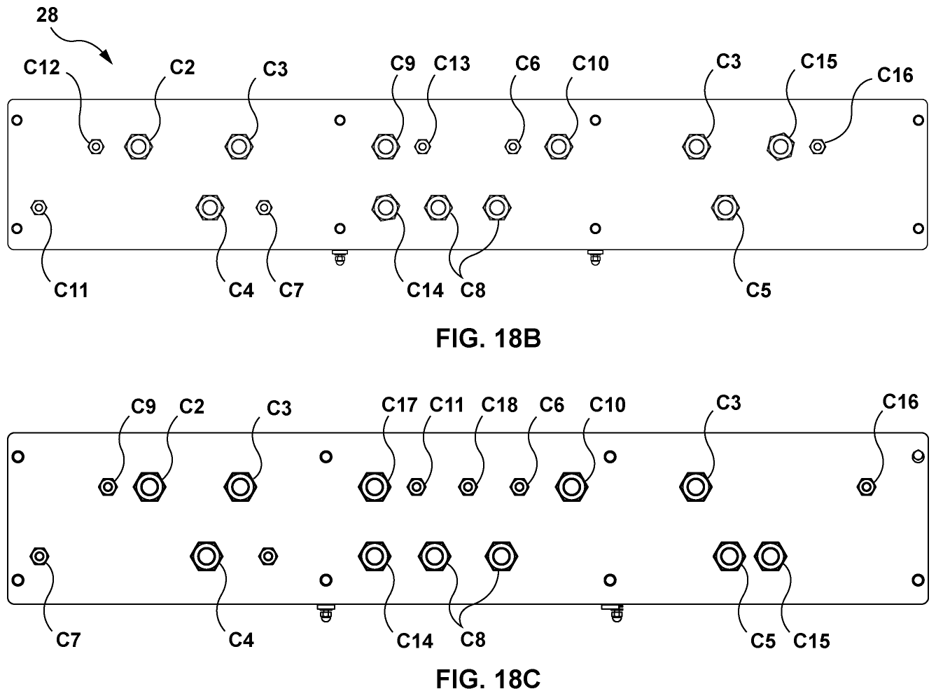

In an embodiment, apparatus 10 may comprise the lower bulkhead plate 28, a connection bulkhead for connecting one or more utilities to apparatus 10. Lower bulkhead plate 28 may comprise the configuration of connections shown in FIG. 2, or a different configuration and/or number or types of connections, such as that of the embodiment shown in FIGS. 8, 10, 18A, and 18B. For example, with reference to FIG. 18B, apparatus 10 may comprise a lower bulkhead plate 28 with connections C2-C10 as described in Table 1, above, and with further connections C11-C16, as shown in FIG. 18B. C11 comprises a low pressure nitrogen feed connection, and C12 and C13 comprise low pressure nitrogen return connections, the nitrogen feed and returns for pressure purging and rendering inert microwave and electrical cabinets, such as cabinets 22b and 68. C15 comprises a coolant supply connection (for coolant supply to cooling mechanism 31 from a chiller external to apparatus 10 (for cooling the coolant)), via cooling mechanism inlet 31a (such as cooling loop inlet 31a) shown in FIGS. 8, 9 and 20C. C14 comprises a coolant return connection (for coolant return from cooling mechanism 31, via cooling mechanism outlet 31b (such as cooling loop outlet 31b) shown in FIGS. 8 and 20B, to the chiller). C16 comprises a connection for supply of compressed air for the actuator valves, for embodiments in which the valves are actuated by compressed air. With reference to FIGS. 9 and 16, apparatus 10 may further comprise feed assembly 70 for supplying the input charge cannabis suspension/solution/resin to reactor tube(s) 12.

In the specific embodiment of cooling mechanism 31 shown in FIG. 20, cooling mechanism inlet 31a may connect to cooling mechanism 31 at connection 31aa (to supply a coolant or cooling solution to the cooling mechanism), and cooling mechanism outlet 31b may connect to cooling mechanism 31 at connection 31bb (to return the coolant or cooling solution to an external cooling source or device). It will be appreciated that the specific arrangement of connections to the cooling mechanism shown is not intended or considered to be limiting on the present invention.

FIG. 18C depicts another example arrangement of connections/ports for a lower bulkhead plate 28 of apparatus 10, comprising connections C2-C10 as described in Table 1, above, C11, C14, C15, and C16 as described above, and with further connections C17 and C18. C17 comprises a drain connection or port, which may be located at a sufficiently low point on apparatus 10 to allow for full drainage of the apparatus for cleaning purposes. C18 comprises a feed relief port as may be required as a drain for a flow meter.

It will be appreciated that the venting of materials from apparatus 10 may be facilitated by any combination of release valve(s) and/or rupture disk(s).

In a further embodiment, the valves described herein may be actuated in an automated fashion based on inputs from the pressure, temperature and/or level sensors, and controls programmed into the control panel/Programmable Logic Controller (PLC) 17. For example, in an embodiment, valves and back pressure regulation may be automatically controlled by controller 17 using HMI 18 (such as by touchscreen controls) to activate the automation, to depressurize the apparatus and render inert its environment (such as the electrical cabinet 54 and microwave cabinet 22b of apparatus 10).

The components described herein may be formed from any suitable material for apparatus 10 (considering the desire to avoid electrical hazards and the potential for reactivity with the transported cannabis suspension/solution/resin). For example, in the example embodiment shown in FIGS. 8 to 10, the following materials may be employed:

TABLE 2
Example Materials for Apparatus Components

| Description | Material |
|---|---|
| Frame assembly 21 | Stainless Steel |
| Microwave Assembly 22a | Stainless Steel |
| Enclosure Assembly 23 | Stainless Steel |
| Upper Manifold 24 | Stainless Steel |
| Holding Pipe 25 | Stainless Steel |
| Nitrogen Feed Assembly | Stainless Steel |
| Nitrogen Out Assembly | Stainless Steel |
| Lower Bulkhead Plate 28 | Stainless Steel |
| Receiver Vessels 29, 30 | Stainless Steel |
| Cooling Mechanism 31 | Stainless Steel |
| Feed assembly to Microwave | Stainless Steel |
| Cooling mechanism/ | Stainless Steel, or copper or any flexible |
| loop outlet 31b | tubing, such as tygon |
| Cooling mechanism/ | Stainless Steel, or copper or any flexible |
| loop inlet 31a | tubing, such as tygon |

Where certain standards govern the operation of apparatus 10, such as the Good Manufacturing Practice (GMP) requirements, apparatus 10 may accordingly be configured for compliance purposes (e.g., compliant materials, such as 316 stainless steel, may be used for pharmaceutical applications of apparatus 10, for GMP compliance). For consumer or natural health products, e.g., other types of materials (e.g., other types of stainless steel) may be used.

In an example embodiment, electrical cabinet 54 may comprise a pressure differential of at least 25 Pa between its pressurized enclosure and the surrounding atmosphere, and apparatus 10 may further comprise a sensor for detecting the nitrogen or other inerting gas level within electrical cabinet 54, which may communicate with controller 17 which in turn, when detecting that the inerting gas level is below a pre-determined/configured threshold amount, may trigger a protective gas supply interruption alarm. It will be appreciated that apparatus 10 may comprise any number of alarms to alert users to dangerous levels detected from sensors of apparatus 10, such as dangerous pressure or temperature levels, as determined by pre-determined/configured thresholds which may be configured via HMI 18. Electrical cabinet 54 may also comprise sensor(s) detecting loss of positive pressure of the protective inert gas within the cabinet. The HMI 18 may be on a screen or panel associated with electrical cabinet 54. For example, controller 17 may be configured (such as by a user via HMI 18) such that when controller 17 receives sensor readings from pressure sensor(s) within electrical cabinet 54 indicating that pressure within the cabinet is below, e.g., 25 Pa, controller 17 may trigger a shutdown of apparatus 10 to avoid operation of the apparatus while the environment around the apparatus 10 electrical components has not been sufficiently purged with an inerting gas (e.g., nitrogen).

Apparatus 10 may be configured for a production room classified as Class 1 Zone 2 of the National Electric Code (NEC). Generally, apparatus 10 may be configured for compliance with applicable regulations, including CAN/CSA C22.2 No. 60079-2-2016, Explosive atmospheres—Part 2: Equipment protection by pressurized enclosure “p”; NFPA 69, Standard on Explosion Prevention Systems; NFPA 496, Standard for Purged and Pressurized Enclosures for Electrical Equipment; NFPA 70, National Electrical Code; NFPA 70E, Standard for Electrical Safety in the Workplace; NFPA 79, Electrical Standard for Industrial Machinery, and to adhere to applicable local and national compliance codes pertaining to installation and operation of equipment in a potentially hazardous environment (e.g., CAN/CSA-C22.2 NO. 157-92 (R2016)—Intrinsically Safe and Non-Incendive Equipment for Use in Hazardous Location).

### 2. Methods of Extraction and Decarboxylation of Cannabis Using the Apparatus

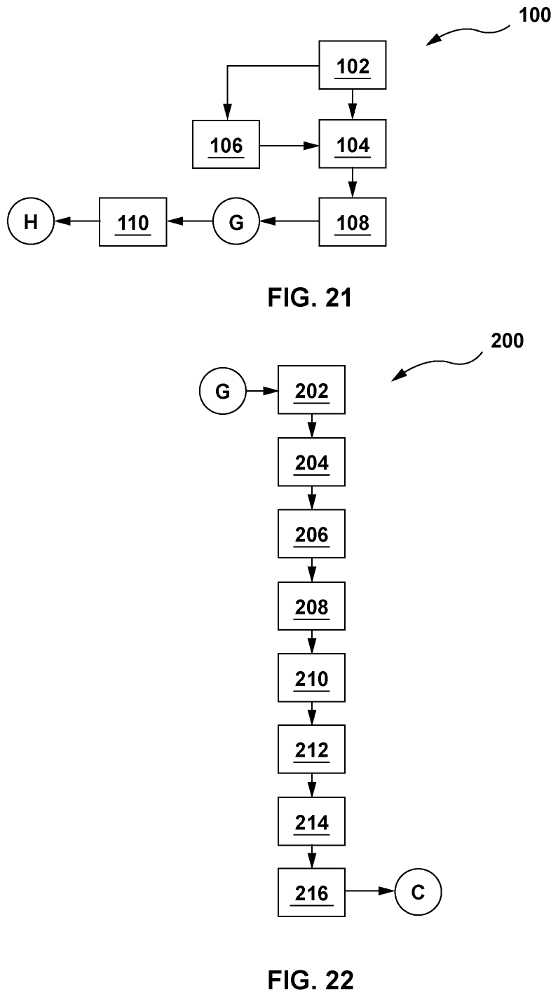

With reference to FIG. 21, method 100 comprises one example method of extraction and decarboxylation of cannabis using apparatus 10, and is described below. Method 100 may comprise: obtaining 102 a cannabis plant material (which may include a hemp variety); combining 104 the cannabis plant material with a solvent (which may comprise ethanol, propanol, or other organic solvent); optionally, grinding 106 the cannabis plant material to extract cannabinoids into the solvent, producing either a cannabis suspension or a cannabis solution; and executing 108 a startup procedure for apparatus 10, comprising at least product flooding and pressurization of the apparatus, in accordance with method 200, as further described hereinafter (method 100 transitions to method 200 at “G”).

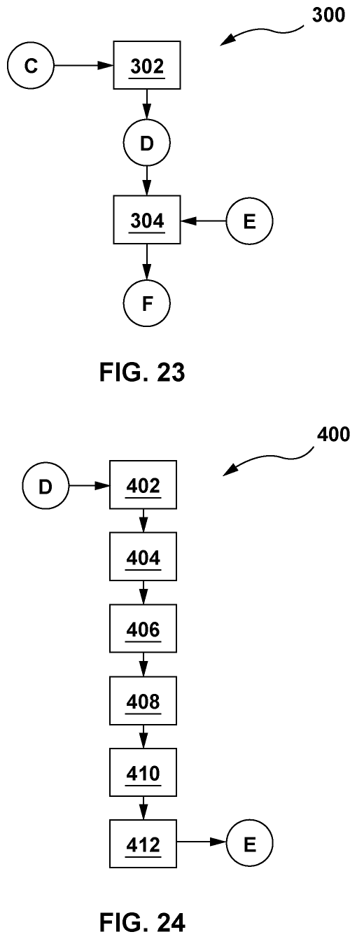

With reference to FIG. 22, apparatus 10 may be charged with the cannabis suspension/solution/resin in an organic solvent following the steps of method 200, including pumping the input materials as well as pressurizing the system, as described below. Method 200 comprises: checking 202 that all safety nuts are intact and tight before using the apparatus; connecting 204 electrical supply to the apparatus; turning on 206 the apparatus; confirming 208 that ventilation is operational (via, e.g., C1; see FIG. 1) to extract chemical fumes in the event of equipment failure; confirming 210 that high and low-pressure nitrogen connections (C6 and C7) are attached and operational; pumping 212 the solvent, such as ethanol or cannabis slurry or cannabis solution, into the apparatus for a sufficient amount of time before system pressurization to ensure the system is flooded with the input prior to pressurization taking place; confirming 214 that valves V9 and V10 are closed; and pressurizing 216 the receiver vessels 29, 30 one at a time via methods 300, 400 and 500, described below. (Apparatus 10 may be pressurized to 140-150 psi (˜10 bar) prior to operation, and as described above, pressure may increase within the apparatus over the course of its operation. Referring to FIGS. 22 and 23, method 200 transitions to method 300 at “C”. Methods 300, 400 and 500, described below, comprise one example set of method steps for sequentially pressurizing the vessels 29, 30.

With reference to FIG. 23, method 300 comprises pressurizing 302 a first receiver vessel 29 via method 400, and pressuring 304 a second receiver vessel 30 via method 500 after the first receiver vessel 29 is pressurized. Method 300 transitions to method 400 at “D”, and to method 500 at “F”.

With reference to FIG. 24, method 400 comprises: applying 402 a high pressure nitrogen source set to slightly higher pressure over the process set point (e.g., 8-10 bar, 116-145 psi); opening 404 the product inlet valve V3 for the first receiver vessel 29; opening 406 the nitrogen feed valve V11 connected to the high pressure nitrogen source for the first receiver vessel 29 slowly pressurizing the first receiver vessel with nitrogen; adjusting 408 the first receiver vessel 29 back pressure regulator valve V5 to suit the set point parameters; when the desired pressure is achieved (e.g., when the pressure in the receiver vessel generally matches the pressure in the tube(s) leading to the receiver vessel) turning off 410 the nitrogen supply; and verifying 412 that product flow is being measured (such as by reference to the HMI 18). Method 400 may then transition to step 304 of method 300 at “E”.

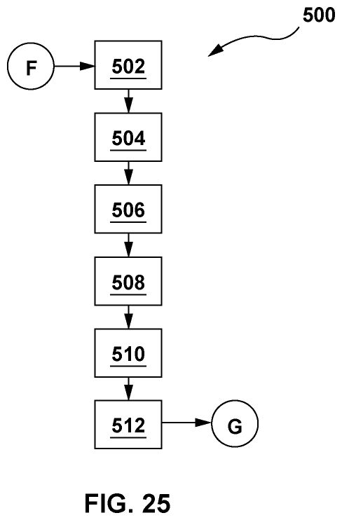

With reference to FIG. 25, method 500 comprises: applying 502 a high pressure nitrogen source set to slightly higher pressure over the process set point; opening 504 the product inlet valve V4 for the second receiver vessel 30; opening 506 the nitrogen feed valve V12 for the second receiver vessel 30 slowly pressurizing the second receiving vessel with nitrogen 30; adjusting 508 the second receiver vessel 30 back-pressure regulator valve V6 to suit the set point parameters; when the desired pressure is achieved (e.g., when the pressure in the receiver vessel generally matches the pressure in the tube(s) leading to the receiver vessel, turning off 510 nitrogen supply; and verifying 512 that product flow is being measured (such as by reference to the HMI 18). “G” in method 500 references “G” in method 100.

In embodiments of apparatus 10 comprising more than two receiver vessels, each vessel will be pressurized sequentially, following a procedure similar that described above for a two-vessel apparatus.

Method 100 may further comprise pumping 110 the cannabis suspension/solution/resin into apparatus 10 and operating the apparatus. Method 100 may transition to method 600 at “H”. Method 600 describes steps for continuous flow operation of apparatus 10, and generally comprises switching collection of the cannabis variety (which may be hemp) product from one receiver vessel to the other receiver vessel to collect the extracted and decarboxylated materials while maintaining operation of apparatus 10.

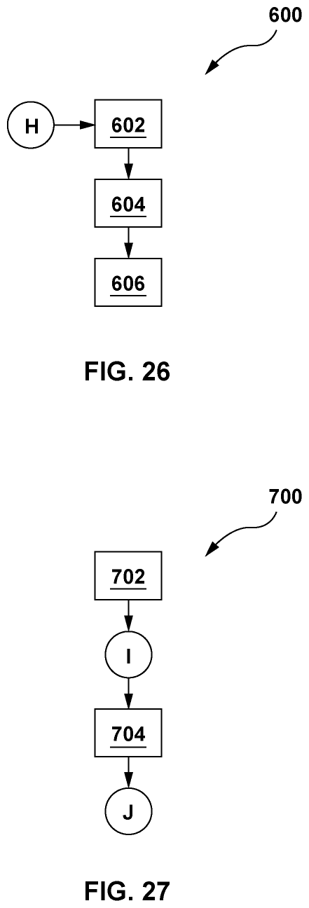

With reference to FIG. 26, method 600, for switching collection of the cannabis variety product from one receiver vessel to the other receiver during operation, comprises: confirming 602 that the second receiver vessel 30 has been pressurized (following method 500, above) to the same or substantially same pressure as the first receiver vessel 29; closing 604 the first receiver vessel product inlet valve V3; and opening 606 the second receiver vessel product inlet valve V4 soon enough after the closing of V3 to prevent a hazardous buildup of pressure in the feed pipeline and possible bursting of the rupture disk(s). Alternatively, the pump inputting the cannabis suspension/solution/resin could be stopped to make the change (should continuous operation not be required, or there is some other need to stop the flow of the material through the apparatus). It will be appreciated that similar steps to that of method 600 would be followed to switch collection from the second receiver vessel 30 to the first receiver vessel 29, accounting for the reversal of valves V3 and V4 in the method steps.

In some embodiments, the valves may be configured to open and close electronically and/or by air pressure, as previously described, and may be configured (such as by the HMI 18) to switch collection from one receiver vessel to the other automatically, upon detection, via sensor(s), of the collected volume of product reaching a predetermined threshold, or the switching of the receiver vessels may be triggered by a user from the control panel 17/HMI 18. Alternatively, or additionally (e.g., in the event of an HMI failure), the valves V4 and V3 may be closed and opened manually. It will be appreciated that any of the valves of the apparatus may be operated in the various manners described herein.

When processing is complete, the receiver vessels may be at a high temperature and pressure (although where a cooling mechanism 31 is used prior to collection in the receiver vessel(s), the temperature and pressure in the receiver pressure(s) is expected to be comparatively reduced). Optionally, it may be confirmed that the cannabis product collected in the receiver vessel(s) is at least 20° C. lower than the product's boiling point before depressurizing the apparatus. For example, ethanol has a boiling point of 78° C. With reference to FIG. 6, where the cannabis suspension/solution/resin is ethanol-based, once the “begin temperature” is less than, e.g., 50° C. the system can be partially depressurized by partially opening valve V7 or V8, depending on the vessel being depressurized. Optionally, the opening of valve V7 or V8 may be automatically controlled by the controller/PLC 17, which may be configured via HMI 18. The residual small amount of pressure may be used to push the product out of the receiver vessel into, e.g., a further collection vessel downstream.

With reference to FIG. 27, method 700 describes steps for releasing pressure from the receiver vessels 29, 30 to collect the cannabis product therefrom. Method 700 comprises: releasing 702 pressure from the first receiver vessel 29 and collecting product therefrom, via method 800; and releasing 704 pressure from the second receiver vessel 30 and collecting product therefrom, via method 900. Method 700 transitions to method 800 at “I”, and to method 900 at “J”.

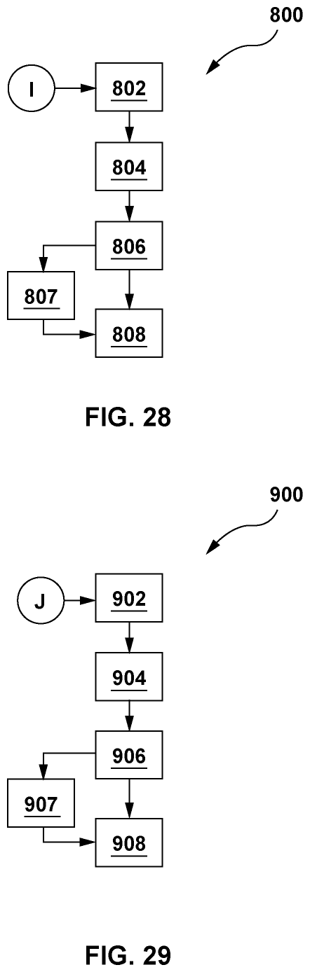

With reference to FIG. 28, method 800 comprises: stopping 802 product flow to the first receiver vessel 29 by closing valve V3; slowly opening 804 valve V7 to begin depressurization of the first receiver vessel 29; confirming 806 that pressure in the first receiver vessel 29 is below a predetermined threshold (e.g., 25 psi) (such as by monitoring the HMI 18 or a pressure gauge); and opening 808 valve V9 to collect the cannabis product via port C4 from the first receiver vessel 29. Optionally, method 800 may comprise confirming 807 that a connection (such as to a collection vessel) is made via C4 prior to carrying out step 808.

With reference to FIG. 29, method 900 comprises: stopping 902 product flow to the second receiver vessel 30 by closing V4; slowly opening 904 valve V8 to begin depressurization of the second receiver vessel 30; confirming 906 that pressure in the second receiver vessel 30 is below a predetermined threshold (e.g., 25 psi) (such as by monitoring the HMI 18 or a pressure gauge); and opening 908 valve V10 to collect the cannabis product via port C5 from the second receiver vessel 30. Optionally, method 900 may comprise confirming 907 that a connection (such as to a collection vessel) is made via C5 prior to carrying out step 908.

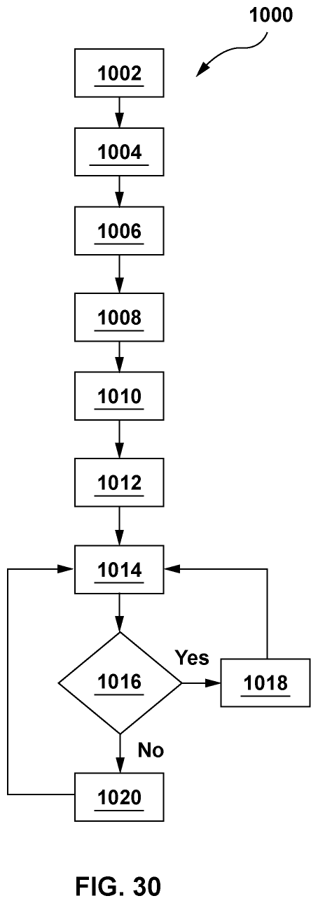

With reference to FIG. 30, method 1000 describes example steps for operating apparatus 10 using an example HMI 18. Method 1000 comprises: logging in 1002 to the HMI using the proper credentials (see FIG. 5 for an example Logon screen of an embodiment of an example HMI 18). During operation, a user may be required to periodically logon (e.g., every 1 hour) to avoid being locked out of the HMI. Method 1000 may further comprise: pressing 1004 the “Fan Start/Stop button”; pressing 1006 the “Waveguide Outlet [which references the reactor tube outlet] Trace Heat” button and set the corresponding temperature; pressing 1008 the “Hold Pipe Trace Heat” button and set the corresponding temperature; pressing 1010 the “Microwave Start/Stop” button (an indicator light, such as a “Microwave ON” light located on the apparatus, such as on the top of the apparatus where it may be easily viewed may be configured to turn on upon activation of the microwave generator 22, to warn users that microwave power has been enabled). Method 1000 may further comprises steps for determining the appropriate microwave power (which microwave tuning steps may be automated), such as: setting 1012 the microwave generator 22 to low microwave power (e.g., 100 W); checking 1014 for reflected microwave power via sensor(s) for detecting reflected microwaves that are communicatively coupled to the HMI; determining 1016 if microwaves are being reflected; if microwaves are being reflected, adjusting 1018 the stub tuner(s) 40 to reduce reflected power; and increasing 1020 microwave power in, e.g., 20 W increments. With each increase of microwave power, steps 1014 to 1018 can be carried out to check for and compensate for microwave reflection at each power level. Tuning operation of the microwave generator 22 may be via auto tuning and controlled remotely. It will be appreciated that controller 17 or HMI 18 may be capable of remote access and control via wired or wireless connection by known means, and a control interface (which may mirror HMI 18) on a remote device, such as desktop computer.

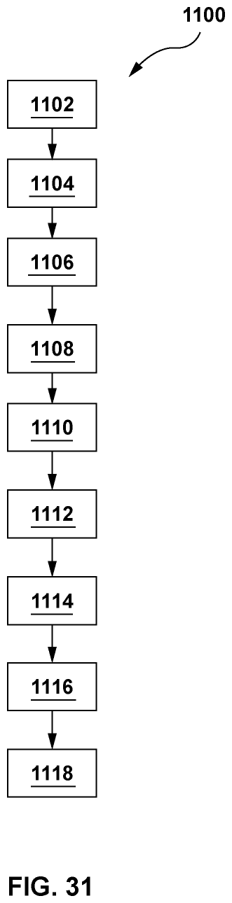

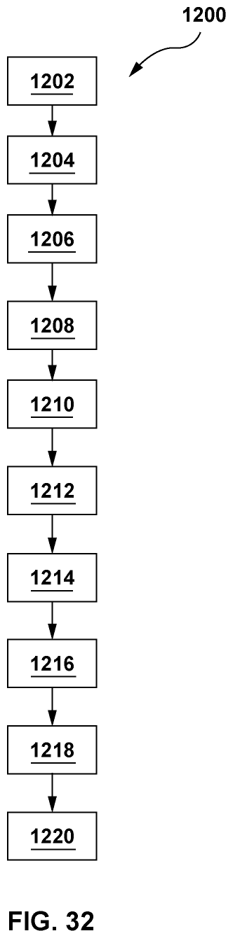

With reference to FIGS. 31 and 32, example methods 1100 and 1200 are described below for extraction and decarboxylation of a cannabis input using apparatus 10. With reference to FIG. 31, method 1100 describes steps for extraction and decarboxylation of a cannabis suspension, and comprises: drying 1102 cannabis plant material; grinding 1104 the dried cannabis plant material to a coarse powder; forming 1106 a cannabis suspension by combining the coarse cannabis powder with a suitable solvent (such as described herein, e.g., ethanol); flowing 1108 under pressure the cannabis suspension through apparatus 10; heating 1110 the cannabis suspension to the desired temperature (e.g., 160° C.) for the desired time to decarboxylate the cannabis suspension; collecting 1112 the decarboxylated cannabis suspension from apparatus 10; filtering 1114 the collected, decarboxylated cannabis suspension (e.g., through an activated carbon filter) to produce a cannabis solution; removing 1116 solvent from the filtered cannabis solution (e.g., by distillation) to obtain a cannabis resin; and drying 1118 the cannabis resin (e.g., by vacuum drying).

With reference to FIG. 32, method 1200 describes steps for extraction and decarboxylation of a cannabis solution, and comprises: drying 1202 cannabis plant material; grinding 1204 the dried cannabis plant material to a coarse powder; forming 1206 a cannabis suspension by combining the coarse cannabis powder with a suitable solvent (such as described herein, e.g., ethanol); forming 1208 a cannabis solution by filtering the cannabis suspension; flowing 1210 under pressure the cannabis solution through apparatus 10; heating 1212 the cannabis solution to the desired temperature (e.g., 160° C.) for the desired time to decarboxylate the cannabis solution; collecting 1214 the decarboxylated cannabis solution from apparatus 10; filtering 1216 the collected, decarboxylated cannabis solution (e.g., through an activated carbon filter); removing 1218 solvent from the filtered cannabis solution (e.g., by distillation) to obtain cannabis a resin and drying 1220 the cannabis resin (e.g., by vacuum drying). Other methods of processing and drying are known to those skilled in the art.

Once the input cannabis suspension/solution/resin is at the desired temperature (150° C., for example) the apparatus may be operated in an Automatic mode in which the controller 17 will regulate the microwave power for heating the reactor tube(s) 12, as well as the trace heating elements or other heating mechanisms around the reactor tube outlet 42b (see FIG. 15) and holding pipe 25 to maintain the cannabis suspension/solution/slurry/resin at set point temperatures after exiting the reactor tube(s). In another embodiment, the step up temperature and microwave tuning steps may also be automated.

The apparatus may be cleaned after completing the extraction, or when desired, depending on the need for such cleaning. An appropriate amount of alcohol (methanol or isopropanol) may be pumped through the apparatus to clean the apparatus. The input to the apparatus may comprise a solution largely devoid of leafy plant material/particulate matter (such as a cannabis suspension/solution that has been strained to reduce the presence of any such leafy material to an acceptable level) to facilitate cleaning of the apparatus tubing/piping. A main process drain provides an outlet at the lowest point of the pipework. The piping system may be designed to allow for the entire system to drain freely, avoiding pooling and dead-legs.

It will be appreciated that while apparatus 10 permits continuous production of extracted and largely or fully decarboxylated cannabis product, in a single pass of the input/charge through the apparatus, apparatus 10 may also be used for batch production of cannabis product by providing the input/charge to the apparatus in batches and/or stopping flow of the charge through the apparatus periodically to collect the cannabis product from the receiver vessel.

Apparatus 10 may comprise a high-capacity apparatus which is expected to be able to provide for large, industrial scale, continuous production of decarboxylated cannabis product (e.g., up to tens of kilograms per hour, or even higher, depending on the dimensions and parameters of the apparatus components, such as the inner/outer diameter of the tubing (or piping) of the apparatus, the length of the tubing, the flow rate at which the input is charged into the apparatus, the number and type of heating mechanisms used to heat and maintain the temperatures, the power output capabilities of the heating mechanism(s) used, number of reactor tubes utilized, and the number and size of receiver vessels, for example). Apparatus 10, depending on various factors such as described above, is expected to be able to achieve production output in the range of 25-50 kg/hr. It will be appreciated that the capacities indicated herein are examples only, and given the scalability of apparatus 10, much higher capacities may be achieved. For example, a version of apparatus 10 may comprise a much larger industrial-sized apparatus, that may occupy much of the floor space in an industrial facility (e.g., 1000 sq. ft.), with numerous receiver vessels and paths thereto operating in parallel, along with multiple reactor tubes appropriately dimensioned for effective and efficient heating, and sufficiently long holding pipes 25 (e.g., 500 m to 1 km long) for achieving complete or substantially complete decarboxylation prior to collection within the receiver vessels. The tubes or pipes leading to/from apparatus 10 may be dimensioned with much larger inner diameters (e.g., ½ foot, 1 foot, etc.), with the number of reactor tubes accordingly being increased to accommodate the increased throughput. It will be appreciated that in such an embodiment, the capacity or throughput capabilities of apparatus 10 would be limited by the ability to supply enough cannabis input rather than by the apparatus itself.

Apparatus 10 comprises materials (such as those described herein) that are capable of withstanding the high pressures to which the apparatus is subjected under normal operating conditions (e.g., 20 bar), and is designed to withstand pressures much higher than those of its normal operating conditions (e.g., up to 1.5 times normal operating pressure, such as up to 30 bar). Such construction is expected to reduce or eliminate the potential for explosion under normal operating conditions, or even where normal operating pressures are exceeded. It will be appreciated that the dimensions and materials of apparatus 10 may be adjusted to accommodate even higher pressures.

Microwave generator 22 may operate with power outputs of between 100 W and 6000 W and at a wavelength between 2425 MHz and 2475 MHz, but it will be appreciated that apparatus 10 may operate at other power outputs and at other wavelengths within the microwave spectrum (i.e., any wavelength from 300 MHz to 300 GHz). An advantage of apparatus 10 in embodiments where microwave generator 22 is utilized is that apparatus 10 is expected to be able to raise the temperature of the cannabis input in a relatively short amount of time, while the input traverses the reactor tube(s) 12, and holding pipe(s) 25 then maintains that temperature which is reached early on in the flow of the input through the apparatus. As such, the cannabis input spends much of its time within apparatus 10 at a sufficiently high temperature to effect complete or substantially complete decarboxylation, to provide a continuous production of the decarboxylated cannabis output in a single pass of the cannabis input through apparatus 10.

Once the decarboxylated cannabis product is collected from one or more of the receiver vessels, it may be subjected to further processing. For example, the extract can be filtered through Celite® and/or activated carbon. The ethanolic extract could also be cooled (winterization) to allow for the precipitation and removal of waxes. The cannabis resin can be isolated by removal of the extraction solvent in vacuo, for example by use of a distillation apparatus or rotary evaporator. Other methods of processing are known to those skilled in the art.

The cannabinoid content of cannabis resin may be characterized by HPLC retention time comparison to qualified reference standards for Δ⁹-THC and other cannabinoids such as Δ⁹-THCA, CBDA, CBGA, CBD, CBG and CBC. The levels of residual solvent remaining in the cannabis resin can be analyzed by GC-MS.

### Example 1

To establish parameters for the apparatus of the present invention, small scale experiments were conducted with a traditional closed batch microwave (i.e. not the apparatus of the present invention). Table 3, below, provides examples of several stir rates, temperatures, exposure times, and reaction pressures used.

TABLE 3

| | Total | Pre- | Stir | Power | Holding | Reaction |
|---|---|---|---|---|---|---|
| Temperature | time | stirring | rate | supplied | Power | Pressure |
| (° C.) | (mins) | (sec) | (rpm) | (W) | (W) | (Bar) |
| 150 | 20 | 30 | 900 | 400 | 60 | 12 |
| 150 | 20 | 30 | 900 | 400 | 60 | 11 |
| 150 | 20 | 30 | 900 | 400 | 60 | 10 |
| 150 | 20 | 30 | 900 | 400 | 55 | 13 |
| 150 | 20 | 30 | 900 | 400 | 60 | 13 |
| 150 | 20 | 30 | 900 | 400 | 60 | 12 |
| 170 | 15 | 30 | 900 | 400 | 70 | 18 |
| 150 | 10 | 30 | 600 | 295 | 38 | 8.5 |
| 150 | 10 | 30 | 600 | 270 | 40 | 8.5 |
| 150 | 10 | 30 | 600 | 360 | 40 | 9 |
| 150 | 10 | 0 | 600 | 400 | 75 | 8 |
| 150 | 10 | 0 | 600 | 400 | 75 | 8.5 |
| 150 | 10 | 0 | 600 | 400 | 75 | 8.5 |
| 150 | 10 | 30 | 600 | 265 | 60 | 8 |
| 150 | 10 | 30 | 600 | 255 | 38 | 9 |
| 150 | 10 | 30 | 600 | 260 | 40 | 8.5 |
| 170 | 10 | 30 | 600 | 310 | 40 | 17 |
| 150 | 10 | 30 | 600 | 280 | 38 | 10 |
| 130 | 10 | 30 | 600 | 250 | 30 | 5 |

### Example 2

Extraction and decarboxylation of Cannabis strain “A” using the apparatus of the present invention

### Experiment 2A:

Milled (ground-up) cannabis (200 g, strain A) was mixed with 95% ethanol (2000 mL). The cannabis slurry/suspension was heated to 60° C. for 2 hours to effect extraction of cannabinoids from the cannabis to the solvent (95% ethanol), then cooled to below 40° C., filtered using a Nutsche™ filter (40 micron), and washed with 95% ethanol (1200 mL) to produce a cannabis solution. The cannabis solution was run through the microwave apparatus using the parameters as indicated in Table 4 to effect decarboxylation. The decarboxylated solution was then cooled and filtered through a bed of activated carbon. The solvent was removed in vacuo to give decarboxylated cannabis resin as a dark brown viscous oil.

### Experiment 2B and 2C:

Milled (ground-up) cannabis (350 g, strain A) was mixed with 95% ethanol (3500 mL). The cannabis slurry/suspension was heated to 60° C. for 2 hours to effect extraction of cannabinoids from the cannabis to the solvent (95% ethanol), then cooled to below 40° C., filtered using a Nutsche™ filter (40 micron), and washed with 95% ethanol (2100 mL) to produce a cannabis solution. The filtrate was then concentrated from 5.6 L to 1.4 L.

2B: A portion of the concentrated cannabis solution (700 mL) was filtered using a Nutsche™ filter (40 micron), washed with ethanol (300 mL), and run through the microwave apparatus using the parameters as indicated in Table 4 to effect decarboxylation. The decarboxylated cannabis solution was then cooled and filtered through a bed of activated carbon. The solvent was removed in vacuo to produce a decarboxylated cannabis resin as a dark brown viscous oil.

2C: A portion the concentrated cannabis solution (700 mL) was filtered using a Nutsche™ filter (40 micron), washed with ethanol (300 mL), and run through the microwave apparatus using the parameters as indicated in Table 4 to effect decarboxylation. The decarboxylated solution was then cooled and filtered through a bed of activated carbon. The solvent was removed in vacuo to produce decarboxylated cannabis resin as a dark brown viscous oil.

### Experiment 2D and 2E:

Milled (ground-up) cannabis (423.6 g, strain A) was mixed with 95% ethanol (3500 mL). The cannabis slurry/suspension was heated to 60° C. for 2 hours to effect extraction, cooled to below 40° C., filtered using a Nutsche™ filter (40 micron), and washed with 95% ethanol (2600 mL) to produce a cannabis solution. The filtrate was then concentrated from 6.0 L to 20 L.

2D: A portion of the concentrated solution (1000 mL) was filtered using a Nutsche™ filter (40 micron), washed with ethanol (100 mL) and run through the microwave apparatus of the present invention using the parameters as indicated in Table 4 to effect decarboxylation. The decarboxylated cannabis solution was then cooled and filtered through a bed of activated carbon. The solvent was removed in vacuo to produce a decarboxylated cannabis resin as a dark brown viscous oil.

2E: A portion the extract (1000 mL) was filtered using a Nutsche™ filter (40 micron), washed with ethanol (100 mL), and run through the microwave apparatus using the parameters as indicated in Table 4 to effect decarboxylation. The decarboxylated solution was then cooled and filtered through a bed of activated carbon. The solvent was removed in vacuo to give cannabis resin as a dark brown viscous oil.

### Experiment 2F:

Milled (ground-up) cannabis (429.5 g, strain A) was mixed with 95% ethanol (3500 mL) to produce a cannabis slurry/suspension). The cannabis slurry/suspension was heated to 60° C. for 2 hours to effect extraction of the cannabinoids from the plant material to the solvent, cooled to below 40° C., filtered using a Nutsche™ filter (40 micron), and washed with 95% ethanol (2100 mL). A portion of the cannabis solution (2000 mL) was run through the microwave apparatus using the parameters as indicated in Table 4 to effect decarboxylation. The decarboxylated solution was then cooled and filtered through a bed of activated carbon. The solvent was removed in vacuo to produce a decarboxylated cannabis resin as a dark brown viscous oil.

### Experiment 2G:

Milled (ground-up) cannabis (4.3 kg, strain A) was mixed with 95% ethanol (34 L) to produce a cannabis slurry/suspension. The slurry/suspension was stirred at room temperature for 4 hours to effect extraction of the cannabinoids from the plant material to the solvent, filtered using a Nutsche™ filter (40 micron), and washed with 95% ethanol (21.5 L). The filtrate was concentrated in vacuo from 55.5 L to 9 L and run through the microwave apparatus using the parameters as indicated in Table 4 to effect decarboxylation. The decarboxylated solution was then cooled and filtered through a bed of activated carbon. The solvent was removed in vacuo to produce a decarboxylated cannabis resin as a dark brown viscous oil.

For experiments 2A-2G, the approximate total run time through the apparatus was 45-540 minutes depending on the flow rate and volume. Since an embodiment of the apparatus is designed as continuous flow, the apparatus can run for extended periods of time, with constant feeding of cannabis suspension/solution/resin. The approximate resident time (the time the solution sat in the holding pipe) was approximately 45-75 minutes. Although dried cannabis was used as the starting material, fresh cannabis may also be used. However, the fresh cannabis has extra weight in the form of water which would need to be removed later.

Filtering the decarboxylated product through a bed of activated carbon is an optional clarification step.

The cannabinoid content of cannabis resin was characterized by HPLC retention time comparison to qualified reference standards for Δ⁹-THC and other cannabinoids such as Δ⁹-THCA, CBDA, CBGA, CBD, CBG and CBC. The results are shown in Table 4. As can be seen, decarboxylation of THCA and CBDA to THC and CBA respectively, was 100% efficient at temperatures of 160° C.

As is known to those skilled in the art, the decarboxylated cannabinoids may be further processed by recovering them from the resin in the form of isolated compounds.

TABLE 4
Data for Microwave Chemistry Reactor Version 1

| | | | End | | | | |
|---|---|---|---|---|---|---|---|
| | Flow | Begin | Setpoint | THC | THCA | CBD | CBDA |
| Experi- | Rate | Temp | Temp | Con- | Con- | Con- | Con- |
| ment | mL/min | ° C. | ° C. | tent | tent | tent | tent |
| 2A | 20 | 150 | 130 | 54% | 0.17% | 0.08% | ND* |
| 2B | 20 | 160 | 140 | 64% | 0.09% | 0.12% | ND |
| 2C | 16 | 160 | 140 | 50.2% | 0.05% | 0.10% | ND |
| 2D | 16 | 160 | 150 | 59% | 0.06% | 0.06% | ND |
| 2E | 16 | 160 | 160 | 61% | ND* | 0.09% | ND |
| 2F | 16 | 160 | 160 | 58% | ND | 0.13% | ND |
| 2G | 21 | 160 | 160 | 68% | ND | 0.11% | ND |

*ND = Not detected.

References made herein to cannabis suspension, solution, resin, or slurry, are intended to cover any variety of the hemp or Cannabaceae family of plants suitable for use with the embodiments of the apparatus and methods described herein, including the Cannabis genus and the Cannabis sativa, Cannabis indica and Cannabis ruderalis spp., as well as varieties with low cannabinoid content (i.e., hemp varieties). It will be appreciated that where it is desired to flow resin through apparatus 10 for further processing, the resin may be mixed with a solvent (such as any of the suitable solvents described herein) for making the resin less viscous so as to facilitate the flow of the resin (in the form of a resin solution) through apparatus 10.

It will be appreciated that any of the functions and method steps described herein may be carried out by a user of apparatus 10 manually, or may be automated by controller/PLC 17, which may be configured by HMI 18 for automation and to set any threshold pressure, level, temperature, etc. settings required to trigger any of the steps described herein. Further, apparatus 10 may be used to effect any chemical reaction requiring high heat and pressure, for a substrate or input passed through the apparatus.

References herein to “automatic” also include semi-automatic operation modes via the HMI 18.

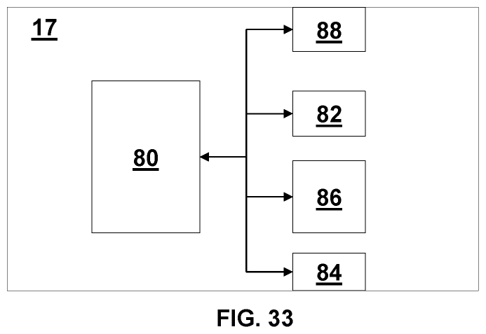

Controller 17 may comprise a computing device (e.g., a laptop computer, desktop computer, tablet, and the like, connected to apparatus 10, or controller 17 may comprise one or more computing, communications, processing, and/or data storage components integrated with apparatus 10). For example, with reference to FIG. 33, controller 17 may comprise one or more processors 80 suitable for carrying out method step(s) described herein and for operation and/or monitoring of components of apparatus 10 (e.g., sensors, valves, etc.). Controller 17 may further comprise or be connected to memory or data storage media 82 (such as hard disks or hard drives, optical storage such as CD or DVD drives, flash storage, RAM (random-access memory), ROM (read-only memory), or any other known means for volatile and/or non-volatile data storage for storage of information for any duration), and may be connected, via communication module 84, and by known means for digital communications, to any type of public or private communications network(s), including LANs (local area networks), WANs (wide area networks), VPNs (virtual private networks), the Internet, or any other type of private or public network of any size, by any known wired and/or wireless communications means (e.g., WiFi™ Ethernet™, Bluetooth™, infrared, WiMAX™, RFID (radio-frequency identification), and any suitable cellular communications protocols, such as GSM, GPRS, EDGE, CDMA, UMTS, LTE, and IMS, and any other communications protocols suitable for the methods and apparatus embodiments described herein, including any proprietary protocols). Controller 17 may interface with or comprise any components necessary to effect communications and/or for network security or operation of apparatus 10, such as routers, firewalls, and access points (not shown). Controller 17 may also comprise a display 86 (which may comprise a touch-screen display), on which HMI 18 may be displayed, as well as one or more input(s) 88 (e.g., a keyboard, mouse, and/or any other known means for providing input to controller 17).

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Numerical data may be presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4 etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

While the foregoing disclosure has been described in some detail for purposes of clarity and understanding, it will be appreciated by one skilled in the art, from a reading of the disclosure that various changes in form and detail can be made without departing from the true scope of the disclosure in the appended claims.

All publications, patents, and patent applications are herein incorporated by reference in their entirety to the same extent as if each individual publication, patent or patent application was specifically and individually indicated to be incorporated by reference in its entirety.

1. An apparatus for extracting and decarboxylating cannabinoids to produce a decarboxylated cannabis product, the apparatus comprising:
one or more inputs; one or more heating tubes;
one or more outputs; and one or more heating mechanisms; the one or more inputs for accepting a cannabis input under pressure, the cannabis input containing the cannabinoids;
wherein, in use, one(s) of said one or more heating mechanisms raises the temperature of the cannabis input to a desired temperature, and other(s) of said one or more heating mechanisms maintains the cannabis input at the desired temperature for a desired time to effect an amount of decarboxylation, and said cannabis input continuously flows from the one or more inputs to the one or more outputs while being subjected to heat by said one or more heating mechanisms at a flow rate sufficient to effect the amount of decarboxylation in a single pass through the apparatus, to produce at said one or more outputs said decarboxylated cannabis product.

2. (canceled)

3. (canceled)

4. (canceled)

5. (canceled)

6. (canceled)

7. (canceled)

8. The apparatus of claim 1 wherein said one(s) of said one or more heating mechanisms comprises a microwave generator.

9. (canceled)

10. (canceled)

11. The apparatus of claim 1 wherein the cannabis input comprises a pharmaceutically acceptable, reagent-grade, food-grade, or pharmaceutical grade organic solvent.

12. The apparatus of claim 11 wherein the organic solvent comprises ethanol or isopropanol.

13. The apparatus of claim 11 wherein the organic solvent has a boiling point less than 100° C.

14. The apparatus of claim 1 wherein the flow rate is between 5 mL/minute and 15 L/minute inclusive.

15. The apparatus of claim 8 wherein one of said heating tubes comprises a reactor tube, wherein said cannabis input is irradiated by microwave radiation to a temperature of from 135° C. to 200° C. inclusive by said microwave generator while traversing said reactor tube.

16. The apparatus of claim 15 wherein said reactor tube comprises quartz.

17. The apparatus of claim 16 wherein the quartz reactor tube has an interior diameter from 4 mm to 120 mm inclusive and a length from 30 cm to 200 cm inclusive.

18. The apparatus of claim 15 further comprising a holding pipe adjacent and contiguous to the reactor tube, said holding pipe for maintaining said cannabis input exiting the reactor tube at said desired temperature, said other(s) of said one or more heating mechanisms for heating the holding pipe.

19. The apparatus of claim 18 wherein said other(s) of said one or more heating mechanisms comprise thermal or infrared radiation.

20. The apparatus of claim 19 wherein said holding pipe is thermally insulated.

21. The apparatus of claim 18 further comprising two or more receiver vessels for collecting said decarboxylated cannabis product from said holding pipe.

22. The apparatus of claim 21 further comprising a cooling mechanism for receiving the cannabis input from the holding pipe to cool the cannabis input to less than 60° C., prior to the cannabis input collection in the two or more receiver vessels.

23. (canceled)

24. (canceled)

25. A method for decarboxylating cannabinoids, comprising passing (a) a suspension of plant material comprising cannabinoids in a solvent or (b) solution of cannabinoids through a continuous flow microwave apparatus, wherein the apparatus heats the suspension or solution to 135° C.-200° C. inclusive under pressure of 10-25 Bar inclusive for sufficient time to decarboxylate the cannabinoids and produce decarboxylated cannabinoids wherein the apparatus heats the suspension or solution under pressure and decarboxylates the cannabinoids to produce decarboxylated cannabinoids.

26. (canceled)

27. (canceled)

28. The method of claim 25, wherein the method further comprises (i) breaking down the plant material before placing the plant material in the solvent to produce the suspension; and (ii) grinding the plant material in the solvent of the suspension, wherein the cannabinoids are extracted from the plant material into the solvent of the suspension during passage through the apparatus.

29. The method of claim 25, wherein the method further comprises a step of processing the decarboxylated cannabinoids into a decarboxylated resin after the step of passing the suspension or solution through the apparatus.

30. The method of claim 25, wherein the resident time of the cannabis suspension or cannabis solution is between 45-75 minutes.

31. The method of any one of claims 25-30, wherein the plant material is selected from the group comprising a cannabis trichome, cannabis female inflorescence, a cannabis flower bract, a cannabis stalk, a cannabis leaf, dried cannabis or a combinations thereof.

32. The method of claim 25 wherein the decarboxylated cannabinoids are recovered in the form of isolated compounds.

33. (canceled)

34. (canceled)

35. (canceled)

36. (canceled)
